SEQUENCE LISTING (3 sequences)

SEQ ID NO: 1 - DNA, 27 bp, synthetic construct
  source 1..27: mol_type = other DNA
acactaagccatccttactgcgcttcg

SEQ ID NO: 2 - DNA, 22 bp, synthetic construct
  source 1..22: mol_type = other DNA
atattgcagcagtacgcacaca

SEQ ID NO: 3 - DNA, 26 bp, synthetic construct
  source 1..26: mol_type = other DNA
acaggtacgttaatagttaatagcgt

(19) United States
(12) Patent Application Publication
(10) Pub. No.: US 2023/0270599 A1
(43) Pub. Date: Aug. 31, 2023

(54) NANOFIBER COLLECTION SWABS

(71) Applicant: Board of Regents of the University of Nebraska, Lincoln, NE (US)

(72) Inventors: Jingwei Xie, Omaha, NE (US); Mark Carlson, Omaha, NE (US); Shixuan Chen, Omaha, NE (US); Alec McCarthy, Omaha, NE (US)

(21) Appl. No.: 18/096,682

(22) Filed: Jan. 13, 2023

Related U.S. Application Data
(60) Provisional application No. 63/299,110, filed on Jan. 13, 2022.

(51) Int. Cl. A61F 13/38; A61B 10/00; A61L 15/32; A61L 15/40; A61L 15/60

(52) U.S. Cl. CPC A61F 13/385; A61B 10/0051; A61L 15/32; A61L 15/40; A61L 15/60; A61L 2400/12

(57) ABSTRACT

Nanofiber swabs are provided as well as methods of use thereof and methods of making.

This application claims priority under 35 U.S.C. § 119(e) to U.S. Provisional Patent Application No. 63/299,110, filed Jan. 13, 2022. The foregoing application is incorporated by reference herein.

This invention was made with government support under Grant No. R01 GM123081 awarded by the National Institutes of Health. The government has certain rights in the invention.

Incorporated herein by reference in its entirety is the Sequence Listing being concurrently submitted as a XML file named SeqList, created Apr. 7, 2023, and having a size of 3,748 bytes.

### FIELD OF THE INVENTION

This application relates to the fields of nanofiber structures. More specifically, this invention provides nanofiber swabs, methods of synthesizing, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

In late 2019, the emergence of the COVID-19 pandemic revealed the importance of swabs in diagnostic testing. The overwhelming shortage of flocked nasopharyngeal swabs led to innovation and repurposing of several new swab types (Vermeiren, et al., J. Clin. Microbiol. (2020) 58: e00669-20; Péré, et al., J. Clin. Microbiol. (2020) 58: e00721-20; Freire-Paspuel, et al., Front. Cell. Infect. Microbiol. (2020) 10:365; Wyllie, et al., N. Engl. J. Med. (2020) 383:1283-1286). Notably, advances in 3D printed swabs of different geometries and repurposing of cotton swabs addressed some of the more immediate shortage needs (Freire-Paspuel, et al., Front. Cell. Infect. Microbiol. (2020) 10:365; Decker, et al., Clin. Infect. Dis. (2021) 73(9): e3027-e3032; Arjunan, et al., J. Mech. Behav. Biomed. Mater. (2021) 114:104175; Ford, et al., 3D Print. Med. (2020) 6:21; Chen, et al., Matter (2020) 3:1589-1600; Callahan, et al., J. Clin. Microbiol. (2020) 58: e00876-20; Williams, et al., Med. J. Aust. (2020) 213:276-279). However, an alarming rate of false negative SARS-CoV-2 tests highlighted the need to not only create more swabs but improve their sample collection yield and recovery to improve testing sensitivity (Pan, et al., Clin. Chem. (2020) 66:794-801; Xiao, et al., J. Med. Virol. (2020) 92(10):1755-1756; West, et al., Mayo Clin. Proc. (2020) 95:1127-1129). Swab performance is largely determined by absorption and release of samples with flocked swabs consistently outperforming cotton and 3D printed swabs in both metrics (Zasada, et al., AMB Express (2020) 10:46; Brownlow, et al., J. Forensic Sci. (2012) 57:713-717; Viviano, et al., Int. J. Women's Health (2018) 10:229-236; Wigger, et al., J. Microbiol. Methods (2019) 157:47-49). Therefore, designing a highly absorptive swab capable of releasing a large percent of the absorbed specimen is a necessity for improved SARS-CoV-2 detection and COVID-19 test sensitivity. Similarly, swabs with higher absorption and release can have direct implications in bacteria, cell, and protein identification and detection, biological sample collection and harvesting, and DNA analysis for forensic applications (Brownlow, et al., J. Forensic Sci. (2012) 57:713-717; Wigger, et al., J. Microbiol. Methods (2019) 157:47-49; Rock, et al., Infect. Control Hosp. Epidemiol. (2018) 39:1257-1261; Probst, et al., Appl. Environ. Microbiol. (2010) 76:5148-5158; Bruijns, et al., J. Forensic Sci. (2018) 63:1492-1499; Webb, et al., J. Mater. Chem. C (2014) 2:10446-10454; Schulz, et al., J. Forensic Sci. (2010) 55:492-498).

### SUMMARY OF THE INVENTION

In accordance with the instant invention, nanofiber swabs are provided along with methods of producing the nanofiber swabs and methods of use. In certain embodiments, the method for producing a nanofiber swab comprises a) fixing (e.g., thermally and/or chemically) at least one point of a nanofiber mat (e.g., a side of a nanofiber mat), b) expanding the nanofiber mat by exposing the nanofiber mat to gas bubbles (optionally by more than one exposure), thereby producing an expanded nanofiber structure, and c) attaching the expanded nanofiber structure to an end of a handle, thereby producing the nanofiber swab. In certain embodiments, the nanofiber mat comprises electrospun nanofibers. In certain embodiments, the expanded nanofiber structure has a rounded geometry (e.g., cylindrical or capsular). In certain embodiments, the expanded nanofiber structure has a rectangular geometry (e.g., rectangular or cubic). In certain embodiments, the gas bubbles are generated as a product of a chemical reaction (e.g., the hydrolysis of sodium borohydride). In certain embodiments, the gas bubbles are generated by a physical releasing (e.g., depressurized CO₂). In certain embodiments, the nanofiber mat is expanded by exposing the nanofiber mat to a subcritical fluid (e.g., subcritical CO₂) and depressurizing (e.g., within a container). In certain embodiments, the nanofiber mat comprises a plurality of aligned nanofibers, random nanofibers, and/or entangled nanofibers. In certain embodiments, the method further comprises synthesizing the nanofiber mat by electrospinning prior to step a). In certain embodiments, the method further comprises cutting and/or trimming the nanofiber mat (e.g., prior to step b) into a desired shape (e.g., rectangle). In certain embodiments, the nanofiber mat is frozen (e.g., with liquid nitrogen) prior to cutting and/or trimming. In certain embodiments, the nanofiber mat comprises polycaprolactone (PCL) and, optionally, a poloxamer (e.g., poloxamer 407 or poloxamer 188). In certain embodiments, the method further comprises coating the expanded nanofiber structure (e.g., before and/or after attachment of the handle) with a substance such as a hydrogel (e.g., gelatin, gelatin methacryloyl (GelMA), and/or chitosan). In certain embodiments, the method further comprises crosslinking the expanded nanofiber structure and/or coating substance (e.g., hydrogel). In certain embodiments, the crosslinking is achieved by contacting and/or exposure to a crosslinker (e.g., glutaraldehyde) or a thermal treatment. In certain embodiments, the handle is attached to the expanded nanofiber structure by an adhesive (e.g., an epoxy).

In accordance with the instant invention, nanofiber swab comprising an expanded nanofiber structure and a handle are provided. In certain embodiments, the expanded nanofiber structure is attached to the terminus of the handle. In certain embodiments, the nanofiber swab is produced by a method of the instant invention.

In accordance with the instant invention, methods of collecting a sample from a surface or subject are provided. In certain embodiments, the method comprises contacting a nanofiber swab with a site in or on the subject. In certain embodiments, the sample is collected from the site via the expanded nanofiber structure. In certain embodiments, the sample is a biological sample. In certain embodiments, the sample comprises a bacteria, a virus, and/or a cell. In certain embodiments, the sample comprises a nucleic acid and/or a polypeptide.

In accordance with the instant invention, the expanded nanofiber structures can be used as wipes (e.g., to clean a surface). In certain embodiments, the expanded nanofiber structure lacks a handle. In certain embodiments, the expanded nanofiber structure is attached to a handle. In certain embodiments, the expanded nanofiber structure is used to remove dust, virus, bacteria, etc. from a surface.

### BRIEF DESCRIPTION OF THE DRAWINGS

FIG. 1A provides a schematic illustration of applications of nanofibers swabs in collecting various biological specimens. FIG. 1B provides a schematic illustration of an example for fabrication of nanofiber swabs including (1) producing uniaxially aligned nanofiber membranes by electrospinning; (2) cutting the membranes into rectangle and fixing one side with thermal treatment; (3) expanding in NaBH₄ solution; (4) coating with gelatin; and (5) bonding to stick to form nanofiber swabs.

FIGS. 2A and 2B provide the characterization and comparison of different swab types. FIG. 2A: Photographs and SEM images of cotton, flocked, and nanofiber swabs.

FIG. 2B: Highly magnified SEM images of cotton, flocked, and nanofiber swabs, comparing micro and nanoscale characteristics of each swab type.

FIGS. 3A-3J provide material characterizations of different swab types. FIG. 3A: Image demonstrating the ability to make different sized nanofiber swabs. FIG. 3B: Porosity of each swab type. FIGS. 3C and 3D: SEM image showcasing macro (FIG. 3C) and microscale (FIG. 3D) pores characteristic of nanofiber swabs' hierarchical structures. FIGS. 3E-3G: SEM images of cotton, flock, and nanofiber swabs' comprising fibers, respectively. FIGS. 3H-3J: Frequency distribution of cotton, flock, and nanofiber swabs' fiber diameters, respectively.

FIG. 4 provides a graph of the mass change of each swab type after vacuum exposure as a mimic for sharp inhalation during nasopharyngeal swabbing (left) and a graph of the mass change of each swab type after 50 and 100 abrasive cycles as a mimic for physical abrasion during nasopharyngeal swabbing (right) (columns from left to right: cotton, flocked and nanofiber swabs).

FIGS. 5A-5E provide a dye absorption analysis. FIG. 5A: Schematic demonstrating the absorbance measuring process. FIG. 5B: Cotton, nanofiber, and flocked swabs before swabbing a dye solution (unloaded), after swabbing a dye solution (swabbed), and after releasing dye (released). Scale bar=2 cm. FIG. 5C: Absorption ratio of each swab type. FIG. 5D: Measured absorbance after releasing absorbed dye. FIG. 5E: Measured release from known volume added to swabs.

FIGS. 6A-6F show protein detection. FIG. 6A: Schematic representation of wet and dry extraction of BSA. FIG. 6B: Picture showing how the moistened swab tips were used to remove dried BSA (dashed circled) from a surface. FIG. 6C: Detected BSA following wet extraction. FIG. 6D: The subsequent percent of protein removed from solution. FIG. 6E: Detected BSA following dry extraction. FIG. 6F: The percent of recovered protein from the surface.

FIGS. 7A-7I show cell and DNA extraction. FIG. 7A: Schematic representation of surface and suspension cell swabbing. FIG. 7B: Extracted cells from suspension swabbing. FIG. 7C: Cells removed from a cell monolayer. FIG. 7D: Microscope images of resuspended cells following surface and suspension swabbing. scale bar=150 μm. FIG. 7E Schematic representation of surface and liquid blood DNA quantification. FIG. 7F: DNA content from known dried blood volumes. FIG. 7G: DNA content from swabbing dried blood droplets (columns from left to right: cotton, flocked and nanofiber swabs). FIG. 7H: Blood absorption ratio of each swab. FIG. 7I: DNA release from each swab tip.

FIGS. 8A-8G show pathogen detection and identification. FIG. 8A: Schematic representation of swabbing for detection of MRSA. FIG. 8B: Plates at three different MRSA titers after extraction using each swab type. FIGS. 8C and 8D: MRSA colony counts at 10⁵ (FIG. 8D) and 10⁴ (FIG. 8C) CFU/ml after swabbing a stock solution. FIG. 8E: Schematic representation from SARS-CoV-2 detection using PCR and swabbing. FIG. 8F: Cycle threshold values at each SARS-CoV-2 titer following swabbing. FIG. 8G: Converted SARS-CoV-2 concentration in pfu/ml (columns from left to right: cotton, flocked and nanofiber swabs).

### DETAILED DESCRIPTION OF THE INVENTION

Following the COVID-19 outbreak, swabs for biological specimen collection were thrust to the forefront of healthcare materials. Swab sample collection and recovery are vital for reducing false negative diagnostic tests, early detection of pathogens, and harvesting DNA from limited biological samples. Herein, a new class of nanofiber swabs are provided which are tipped with hierarchical 3D nanofiber objects produced by expanding electrospun membranes with a solids-of-revolution-inspired gas foaming technique. Nanofiber swabs significantly improve absorption and release of proteins, cells, bacteria, DNA, and viruses from solutions and surfaces. Implementation of nanofiber swabs in SARS-CoV-2 detection reduces the false negative rates at two viral concentrations and identifies SARS-CoV-2 at a 10× lower viral concentration compared to flocked and cotton swabs. The nanofiber swabs will improve test sensitivity, leading to timely and accurate diagnosis of many diseases.

In accordance with the instant invention, nanofiber swabs, methods of synthesizing nanofiber swabs, and method of using nanofiber swabs are provided. As described herein, the nanofiber swabs of the instant invention have improved collection efficiency over other swabs including traditional cotton and flocked swabs. In certain embodiments, the nanofiber swab is a specimen collection swab. The swabs of the instant invention can be used to collect specimen from anywhere, including any part of a subject. In certain embodiments, the nanofiber swab is a nasal swab. In certain embodiments, the nanofiber swab is a nasopharyngeal swab. In certain embodiments, the nanofiber swab is an oropharyngeal swab.

These nanofiber swabs of the instant invention may be used to collect biological and/or non-biological specimens including but not limited to: fluids (e.g. blood, saliva, urine, serum, plasma, etc.), exudates, bacteria, viruses, cells and/or subcellular materials (e.g., nucleic acid molecules, DNA, RNA, proteins, peptides, polypeptides, etc.). The nanofiber swabs of the instant invention may be used in medicine. The nanofiber swabs of the instant invention may be used, without limitation, in clinical, veterinarian, forensic, agriculture, and/or any non-clinical settings. The nanofiber swabs may be used to collect a specimen from a non-biological entity such as a surface at a crime scene.

Generally, the nanofiber swabs of the instant invention comprise an expanded, nanofiber structure comprising a plurality of nanofibers. The expanded, nanofiber structure of the instant invention (e.g., the head or tip of the nanofiber swab) may be attached to a handle. The expanded, nanofiber structure will typically be at the end or tip of the swab handle. In certain embodiments, the expanded, nanofiber structure covers or encompasses the end or tip of the swab handle. In certain embodiments, the nanofiber swab comprises an expanded, nanofiber structure at each end of the handle (i.e., the nanofiber swab comprises two expanded, nanofiber structures). When the nanofiber swab comprises two expanded, nanofiber structures, the first and second expanded, nanofiber structures can be the same or different (e.g., different in size, and/or different polymer, etc.).

The swab handle may be made from a variety of materials. For example, the swab handle may comprise, without limitation: wood, plastics and other polymers, paper (e.g., rolled paper) and paper related materials, and/or metal. In certain embodiments, the swab handle comprises plastic. Generally, the swab handle is rounded or has a rounded geometry (e.g., a cylinder, cylindrical, tubular, etc.).

The nanofiber material may be assembled directly onto the swab handle or the nanofiber material may be attached to the swab handle (e.g., via an adhesive agent). For example, the swab handle may be inserted into the expanded, nanofiber structure (e.g., in to the middle (e.g., into a cavity in the middle created by the expansion process)). The swab handle may be attached or fixed to the expanded, nanofiber structure. The swab handle may be attached or fixed by any means. In certain embodiments, the expanded, nanofiber structure is mechanically attached or fixed to the end of the swab handle. In certain embodiments, an adhesive (e.g., epoxy, glue, cement, paste, binder, etc.) is used to attach the expanded, nanofiber structure to the swab handle. In certain embodiments, the expanded, nanofiber structure is attached to the swab handle by an epoxy.

The swab handle can be any length. In certain embodiments, the swab handle is less than about 25 cm in length, less than about 22 cm in length, less than about 20 cm in length, less than about 18 cm in length, less than about 16 cm in length, less than about 14 cm in length, less than about 12 cm in length, less than about 10 cm in length, or less than about 8 cm in length. In certain embodiments, the swab handle is more than about 4 cm in length, more than about 6 cm in length, more than about 8 cm in length, more than about 10 cm in length, more than about 12 cm in length, more than about 14 cm in length, more than about 16 cm in length, more than about 18 cm in length, more than about 20 cm in length, more than about 22 cm in length, or more than about 24 cm in length. In certain embodiments, the swab handle is about 6 cm to about 25 cm in length, about 6 cm to about 22 cm in length, about 8 cm to about 20 cm in length, about 10 cm to about 20 cm in length, about 12 cm to about 20 cm in length, about 12 cm to about 18 cm in length, or about 14 cm to about 16 cm in length. In certain embodiments, the swab handle is about 1 mm to about 10 mm, about 1 mm to about 5 mm, about 2 mm to about 5 mm, or about 2 mm to about 4 mm in thickness (e.g., average diameter).

The nanofiber swabs of the present invention can be formed and manufactured into any shape, size, and/or thickness. For example, the nanofiber swab may have a three dimensional shape such as, without limitation: a capsule, cylinder, tube, cone, sphere, dome, rectangle, or cube. In certain embodiments, the nanofiber swab has a rounded geometry. For example, the nanofiber swabs may be spherical, cylindrical, or capsular in shape. Generally, a capsular shape approximates the shape of a capsule (e.g., a geometric shape consisting of a cylinder with hemispherical ends). In certain embodiments, the expanded nanofiber structure has a rectangular geometry (e.g., rectangular or cubic).

The nanofibers of the instant invention can be fabricated by any method. For example, the nanofiber material may be manufactured using a variety of methods including but not limited to electrospinning, phase separation, centrifugal force spinning, hypersonic spinning, and aerogels. In certain embodiments, the nanofiber mat or structure is synthesized by electrospinning. In certain embodiments, the expanded, nanofiber structures comprise electrospun nanofibers. The expanded nanofiber structure may comprise aligned fibers (e.g., uniaxially aligned), random fibers, and/or entangled fibers. In certain embodiments, the expanded nanofiber structure comprises aligned fibers (e.g., uniaxially, radially, vertically, or horizontally). In certain embodiments, the expanded nanofiber structure comprises radially aligned nanofibers, vertically aligned nanofiber, or a combination thereof. While the application generally describes nanofibers (fibers having a diameter less than about 1 m (e.g., average diameter)) structures and the synthesis of three-dimensional nanofibrous structures, the instant invention also encompasses microfibers (fibers having a diameter greater than about 1 m (e.g., average diameter)) structures and the synthesis of three-dimensional microfibrous structures.

In certain embodiments of the instant invention, the methods comprise fixing at least one point, edge, end, or side—or a portion thereof—of a nanofiber mat (sometimes referred to as 2D structure herein) and then expanding the nanofiber mat into an expanded nanofiber structure (sometimes referred to as a 3D scaffold herein). In certain embodiments, a whole or entire side of the nanofiber mat is fixed. In certain embodiments, one or more sections or portions of the nanofiber mat is fixed (e.g., the top and bottom corners on one side may be fixed). The nanofiber mat may be fixed by any means. For example, the nanofiber mat may be thermally fixed or chemically fixed. In certain embodiments, the nanofiber mat is thermally fixed.

In certain embodiments, the nanofiber mat is fixed by exposing at least one point, edge, end, or side—or a portion thereof—of the nanofiber mat to elevated temperatures. In certain embodiments, the nanofiber mat is exposed to temperatures at or above the melting temperature of the nanofibers. In certain embodiments, the nanofiber mat is fixed by exposing at least one point, edge, end, or side—or a portion thereof—of the nanofiber mat to a temperature of at least about 50° C., 55° C., 60° C., 65° C., 70° C., 75° C., 80° C., 85° C., 90° C., 95° C., 100° C., or higher. To avoid excess fixation and/or damage to the remainder of the nanofiber mat, the exposure to elevated temperatures may be brief (e.g., less than 10 seconds, less than 5 seconds, or for about 1 second). In certain embodiments, the heat is applied perpendicularly to the nanofiber mat. In certain embodiments, the thermal fixing comprises exposing at least one point, edge, end, or side—or a portion thereof—of a nanofiber mat to about 75° C. to about 95° C., particularly about 85° C. (e.g., for less than 5 seconds, particularly about 1 second).

In certain embodiments, the nanofiber mat is chemically fixed, for example, by exposure to a chemical, solvent, or crosslinker. In certain embodiments, a chemical or solvent based method is used to fix the nanofiber mat. The chemical or solvent can be, without limitation: dichloromethane (DCM), dimethylformamide (DMF), dichloroformamide, acetone, and other organic solvents. In certain embodiments, the nanofiber mat is fixed by exposure to a crosslinker. In certain embodiments, the nanofiber mat is chemically fixed by exposing at least one point, edge, end, or side—or a portion thereof—of the nanofiber mat to a chemical, solvent, or crosslinker with minimal or no exposure the remainder of the nanofiber mat to the chemical, solvent, or crosslinker.

The methods of the instant invention may further comprise synthesizing the nanofibrous structure (e.g., mat) prior to expansion (e.g., exposure to gas bubbles). In certain embodiments, the nanofiber mat is synthesized using electrospinning. In certain embodiments, the nanofiber mat comprises aligned fibers (e.g., uniaxially), random fibers, and/or entangled fibers.

The nanofiber mat may be cut, trimmed, or shaped prior to expansion. The nanofiber mat may be cut, trimmed, or shaped prior to fixation or cut, trimmed, or shaped after fixation. In certain embodiments, the nanofiber mat is cut, trimmed, or shaped under cryogenic or frozen conditions (e.g., in liquid nitrogen). The nanofiber mat can be cut, trimmed, or shaped into any desired shape such as, without limitation: rectangles, squares, triangles, quadrangles, pentagons, hexagons, circles, ovals, semicircles, L's, C's, O's, U's, and arches. While the application generally describes nanofiber mats as the 2D structure prior to expansion, the instant invention also encompasses any nanofibrous structure which can be expanded by the methods provided herein (e.g., structures other than a mat or 3D structures which can be further expanded).

In certain embodiments, the nanofiber mat is synthesized (e.g., electrospun) as a rectangle or square. In certain embodiments, the nanofiber mat is cut into a rectangle or square. In certain embodiments, the nanofiber mat is rectangular. In certain embodiments, the width (e.g., average width) of the nanofiber mat is less than 10 mm, less than 9 mm, less than 8 mm, less than 7 mm, less than 6 mm, less than 5 mm, less than 4 mm, less than 3 mm, less than 2 mm, or less than 1 mm. In certain embodiments, the width (e.g., average width) of the nanofiber mat is more than 1 mm, more than 2 mm, more than 3 mm, more than 4 mm, more than 5 mm, more than 6 mm, more than 7 mm, more than 8 mm, or more than 9 mm. In certain embodiments, the width (e.g., average width) of the nanofiber mat is about 0.5 mm to about 10 mm, about 0.5 mm to about 8 mm, about 0.5 mm to about 6 mm, about 0.5 mm to about 5 mm, about 1 mm to about 5 mm, about 1 mm to about 4 mm, about 2 mm to about 4 mm, or about 2 mm to about 3 mm. In certain embodiments, the length (e.g., average length) of the nanofiber mat is more than about 3 mm, more than about 5 mm, more than about 6 mm, more than about 7 mm, more than about 8 mm, more than about 9 mm, more than about 10 mm, more than about 11 mm, more than about 12 mm, more than about 13 mm, more than about 14 mm, more than about 15 mm, more than about 16 mm, more than about 17 mm, more than about 18 mm, more than about 19 mm, more than about 20 mm, more than about 21 mm, more than about 22 mm, more than about 23 mm, more than about 24 mm, more than about 25 mm, more than about 26 mm, more than about 27 mm, more than about 28 mm, more than about 29 mm, more than about 30 mm, or more than about 35 mm. In certain embodiments, the length (e.g., average length) of the nanofiber mat is less than about 40 mm, less than about 35 mm, less than about 30 mm, less than about 29 mm, less than about 28 mm, less than about 27 mm, less than about 26 mm, less than about 25 mm, less than about 24 mm, less than about 23 mm, less than about 22 mm, less than about 21 mm, less than about 20 mm, less than about 19 mm, less than about 18 mm, less than about 17 mm, less than about 16 mm, less than about 15 mm, less than about 14 mm, less than about 13 mm, less than about 12 mm, less than about 11 mm, less than about 10 mm, less than about 9 mm, less than about 8 mm, less than about 7 mm, less than about 6 mm, less than about 5 mm, or less than about 3 mm. In certain embodiments, the length (e.g., average length) of the nanofiber mat is about 3 mm to about 40 mm, about 5 mm to about 30 mm, about 5 mm to about 25 mm, about 10 mm to about 30 mm, about 10 mm to about 20 mm, about 12 mm to about 20 mm, about 12 mm to about 18 mm, or about 12 mm to about 15 mm.

In certain embodiments, the area (e.g., surface area) of the nanofiber mat is about 10 mm² to about 200 mm², about 10 mm² to about 100 mm², about 10 mm² to about 75 mm², about 20 mm² to about 75 mm², about 20 mm² to about 50 mm², about 25 mm² to about 55 mm², or about 25 mm² to about 45 mm². In certain embodiments, the area (e.g., surface area) of the nanofiber mat is less than about 200 mm², less than about 150 mm², less than about 125 mm², less than about 100 mm², less than about 90 mm², less than about 80 mm², less than about 70 mm², less than about 60 mm², less than about 50 mm², less than about 40 mm², or less than about 30 mm². In certain embodiments, the area (e.g., surface area) of the nanofiber mat is more than about 1 mm², more than about 5 mm², more than about 10 mm², more than about 15 mm², more than about 20 mm², more than about 25 mm², or more than about 30 mm².

In certain embodiments, the thickness (e.g., average thickness) of the nanofiber mat is about 0.01 mm to about 10 mm, about 0.05 to about 10 mm, about 0.1 to about 10 mm, about 0.1 to about 5 mm, about 0.1 to about 3 mm, about 0.5 to about 3 mm, about 0.5 to about 2 mm, about 0.75 to about 1.5 mm, or about 0.75 to about 1.25 mm. In certain embodiments, the thickness (e.g., average thickness) of the nanofiber mat is more than about 0.01 mm, more than about 0.025 mm, more than about 0.05 mm, more than about 0.75 mm, more than about 0.1 mm, more than about 0.2 mm, more than about 0.3 mm, more than about 0.4 mm, more than about 0.5 mm, more than about 0.6 mm, more than about 0.7 mm, more than about 0.8 mm, more than about 0.9 mm, more than about 1 mm, more than about 2 mm, more than about 3 mm, more than about 4 mm, or more than about 5 mm. In certain embodiments, the thickness (e.g., average thickness) of the nanofiber mat is less than about 20 mm, less than about 17.5 mm, less than about 15 mm, less than about 12.5 mm, less than about 10 mm, less than about 9 mm, less than about 8 mm, less than about 7 mm, less than about 6 mm, less than about 5 mm, less than about 4 mm, less than about 3 mm, less than about 2 mm, or less than about 1 mm.

In certain embodiments, the nanofiber mat is expanded into an expanded nanofiber structure by exposing the nanofiber mat to gas bubbles. In certain embodiments, the nanofiber mat is expanded radially and/or around a fixed axis (e.g., as defined by the fixed portion of the nanofiber mat). The bubbles can be generated by chemical reactions or physical manipulations. For example, the nanofiber mat can be submerged or immersed in a bubble/gas producing chemical reaction or physical manipulation. Generally, the longer the exposure to the bubbles, the greater the thickness and porosity of the expanded nanofiber structure increases. The nanofiber mat may also be expanded within a mold (e.g., a metal, plastic, or other material that does not expand in the presence of gas bubbles) to assist in the formation of a desired shape. The nanofiber mat may be treated with air plasma prior to exposure to gas bubbles (e.g., to increase hydrophilicity). The nanofiber mat may be exposed to gas bubbles more than once (e.g., repeatedly).

After exposure to the bubbles, the expanded nanofiber structure may be washed and/or rinsed in water and/or a desired carrier or buffer (e.g., a pharmaceutically or biologically acceptable carrier). Trapped gas bubbles may be removed by applying a vacuum to the expanded nanofiber structure. For example, the expanded nanofiber structure may be submerged or immersed in a liquid (e.g., water and/or a desired carrier or buffer) and a vacuum may be applied to rapidly remove the gas bubbles. The process may be repeated one or more times. After expansion (e.g., after rinsing and removal of trapped gas), the expanded nanofiber structure may be placed in storage in cold solution or lyophilized and/or freeze-dried.

The gas bubbles of the instant invention can be made by any method known in the art. The bubbles may be generated, for example, by chemical reactions or by physical approaches. Electrospun nanofiber mats can be expanded three dimensionally using a gas foaming based expansion method (example methods may be found in WO 2016/053988; WO 2019/060393; and WO 2020/124072, each incorporated herein by reference). Electrospun nanofiber mats can be expanded in the third dimension with ordered structures using gas bubbles generated by chemical reactions in an aqueous solution (see, e.g., WO 2016/053988; WO 2019/060393; Jiang et al. (2018) Acta Biomater., 68:237-248; Jiang, et al. (2015) ACS Biomater. Sci. Eng., 1:991-1001; Jiang, et al. (2016) Adv. Healthcare Mater., 5:2993-3003; Joshi, et al. (2015) Chem. Eng. J., 275:79-88; each of the foregoing incorporated by reference herein). In certain embodiments, the chemical reaction or physical manipulation does not damage or alter or does not substantially damage or alter the nanofibers (e.g., the nanofibers are inert within the chemical reaction and not chemically modified). As explained hereinabove, the nanofiber mat may be submerged or immersed in a liquid comprising the reagents of the bubble-generating chemical reaction. Examples of chemical reactions that generate bubbles include, without limitation:

NaBH₄+2H₂O═NaBO₂+4H₂

NaBH₄+4H₂O=4H₂(g)+H₃BO₃+NaOH

HCO₃⁻+H⁺=CO₂+H₂O

NH₄⁺+NO₂=N₂+2H₂O

H₂CO₃=H₂O+CO₂

2H⁺+S²⁻═H₂S

2H₂O₂=O₂+2H₂O

3HNO₂=2NO+HNO₃+H₂O

HO₂CCH₂COCH₂CO₂H=2CO₂+CH₃COCH₃

2H₂O₂=2H₂+O₂

CaC2+H₂O═C₂H₂

Zn+2HCl=H₂+ZnCl₂

2KMnO₄+16HCl=2KCl+2MnCl₂+H₂O+5Cl₂

In certain embodiments, the chemical reaction is the hydrolysis of NaBH₄ (e.g., NaBH₄+2H₂O═NaBO₂+4H₂). In certain embodiments, CO₂ gas bubbles (generated chemically or physically) are used (e.g., for hydrophilic polymers).

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use subcritical gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber mat may be submerged or immersed in a liquid of the bubble-generating physical manipulation.

In certain embodiments, the nanofiber mats are expanded using a subcritical or supercritical fluid or liquid (e.g., CO₂, N₂, N₂O, hydrocarbons, and fluorocarbons). In certain embodiments, the nanofiber mats are expanded by exposure to depressurized CO₂. In certain embodiments, liquid CO₂ is utilized. For example, nanofiber mats may be expanded by exposing to, contacting with or being placed into (e.g., submerged or immersed) a subcritical liquid/fluid (e.g., subcritical CO₂) and then depressurized. The cycle of placing the nanofibrous structures into subcritical CO₂ and depressurizing may be performed one or more times. Generally, the more times the expansion method is used the thickness and porosity of the nanofibrous (or microfibrous) structure increases. For examples, the cycle of exposure to subcritical CO₂ and then depressurization may be performed one, two, three, four, five, six, seven, eight, nine, ten, or more times, particularly 1-10 times, 1-5 times, or 1-3 times. In certain embodiments, the cycle of exposure to subcritical CO₂ and then depressurization is performed at least 2 times (e.g., 2-10 times, 2-5 times, 2-4 times, or 2-3 times). In certain embodiments, the method comprises placing the nanofibrous mat and dry ice (solid CO₂) in a sealed container, allowing the dry ice to turn into liquid CO₂, and then unsealing the container to allow depressurization.

The nanofiber mat and subcritical fluid (e.g., subcritical CO₂; or solid form of subcritical fluid (e.g., dry ice)) may be contained in any suitable container (e.g., one which can withstand high pressures). For example, the subcritical fluids and the nanofiber mat may be contained within, but not limited to: chambers, vessels, reactors, and tubes. In certain embodiments, the equipment or container used during the methods of the present invention will have a feature or component that allows control of the depressurization rate of the subcritical fluid. Depressurization of the subcritical fluid can be done using a variety of methods including but not limited to manually opening the container to decrease pressure or by using some type of equipment that can regulate the rate of depressurization of the reaction vessel.

The nanofibers of the instant invention may comprise any polymer. In certain embodiments, the polymer is biocompatible. The polymer may be biodegradable or non-biodegradable. In certain embodiments, the polymer is a biodegradable polymer. The polymer may by hydrophobic, hydrophilic, or amphiphilic. In certain embodiments, the polymer is hydrophobic. In certain embodiments, the polymer is hydrophilic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium alginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In certain embodiments, the nanofiber comprises polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, chitosan, starch, pectin, cellulose, methylcellulose, sodium polyacrylate, starch-acrylonitrile co-polymers, and/or combinations of two or more polymers. In certain embodiments, the polymer comprises polycaprolactone (PCL). In certain embodiments, the polymer comprises polycaprolactone (PCL) and gelatin (e.g., at a 1:1 ratio).

In certain embodiments, the nanofiber, nanofiber mat, and/or expanded nanofiber structure may further comprise at least one amphiphilic block copolymer comprising hydrophilic poly(ethylene oxide) (PEO) and hydrophobic poly(propylene oxide) (PPO). In certain embodiments, the nanofiber mat and/or expanded nanofiber structure comprises a poloxamer or an amphiphilic triblock copolymer comprising a central hydrophobic PPO block flanked by two hydrophilic PEO blocks (i.e., an A-B-A triblock structure). In certain embodiments, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In certain embodiments, the nanofiber, nanofiber mat, and/or expanded nanofiber structure comprises poloxamer 188. In certain embodiments, the nanofiber, nanofiber mat, and/or expanded nanofiber structure comprises poloxamer 407 (Pluronic® F127). The amphiphilic block copolymer (e.g., poloxamer) may be added in various amounts to the polymer solution during the synthesis process (e.g., electrospinning). In certain embodiments, about 0% to about 20%, about 0% to about 15%, about 0% to about 10%, about 0.1% to about 5%, about 0.5% to about 2%, or about 0.1% to about 1.0% (e.g., w/v) of the polymer solution is an amphiphilic block copolymer (e.g., a poloxamer (e.g., poloxamer 407 and/or poloxamer 188)). In certain embodiments, about 0.1% to about 50%, about 0.1% to about 40%, about 0.1% to about 30%, about 0.1% to about 25%, about 0.1% to about 20% (e.g., w/v) of the polymer solution is polymer (e.g., PCL).

In certain embodiments, the polymer solution comprises about 10% polymer (w/v) (e.g., PCL) and about 0.5% poloxamer 407 (w/v) (Pluronic® F127). In certain embodiments, the polymer solution comprises PCL and poloxamer 407 in a ratio (e.g., by weight) of about 200:1 to about 2:1, about 100:1 to about 4:1, about 50:1 to about 8:1, about 40:1 to about 10:1, about 30:1 to about 15:1, or about 20:1.

In certain embodiments, the nanofibers and/or nanofiber structures are coated with additional materials to enhance their properties. In certain embodiments, the expanded nanofiber structure, optionally attached to the handle, is coated with the additional material. For example, the nanofibers and/or nanofiber structure can be coated with a material to help reinforce the structure and increase fluid absorption. In certain embodiments, the nanofibers and/or nanofiber structure may be coated with proteins, collagen, fibronectin, collagen, a proteoglycan, elastin, or a glycosaminoglycans (e.g., hyaluronic acid, heparin, chondroitin sulfate, or keratan sulfate). In certain embodiments, the nanofibers and/or nanofiber structures comprise a material that enhances the nanofiber structure's ability to absorb fluids, particularly aqueous solutions (e.g., blood), and/or allow for the 3D shapes/structures of the expanded nanofiber structure to be recoverable after compression. In certain embodiments, the nanofibers comprise a polymer and the material which enhances the absorption properties.

In certain embodiments, the nanofibers and/or nanofiber structures are coated with the material which enhances the absorption properties. The term “coat” refers to a layer of a substance/material on the surface of a structure. Coatings may, but need not, also impregnate the nanofiber structure. Further, while a coating may cover 100% of the nanofibers and/or nanofiber structure, a coating may also cover less than 100% of the surface of the nanofibers and/or nanofiber structure (e.g., at least about 75%, at least about 80%, at least about 85%, at least about 90%, at least about 95%, at least about 98%, or more the surface may be coated). Materials which enhance the absorption properties of the expanded nanofiber structures include, without limitation: gelatin, gelatin methacryloyl (GelMA), alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In certain embodiments, the material is a hydrogel (e.g., a polymer matrix able to retain water, particularly large amounts of water, in a swollen state). In certain embodiments, the material is gelatin, gelatin methacryloyl (GelMA), and/or chitosan. In certain embodiments, the material is gelatin. In certain embodiments, the expanded nanofiber structure is coated with the coating material (e.g., gelatin) by submersion into a solution of the coating material (e.g., gelatin). In certain embodiments, the expanded nanofiber structures are coated with about 0.05% to about 20%, about 0.1% to about 20%, about 0.1% to about 10%, or about 0.1% to about 1% coating material (e.g., gelatin) (e.g., by weight or w/v). In certain embodiments, the coating material (e.g., hydrogel) is freeze-dried after coating. In certain embodiments, the coating material (e.g., hydrogel) is crosslinked (e.g., by glutaraldehyde) after coating (e.g., after freeze-drying).

In certain embodiments, the nanofiber structures of the instant invention are crosslinked (e.g., before or after expansion). In certain embodiments, the expanded nanofiber structure is crosslinked after coating. Crosslinking may be done using a variety of techniques including thermal crosslinking, chemical crosslinking, UV-crosslinking, and photo-crosslinking. For example, the nanofiber structures of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In certain embodiments, the crosslinker is glutaraldehyde. In certain embodiments (e.g., instead of crosslinking or in addition to crosslinking), the nanofiber structure is thermally treated (e.g., at a temperature close to, but below, the melting point of the nanofibers; e.g., about 50° C.).

The expanded nanofiber structures and/or nanofiber swabs of the instant invention may also be sterilized. For example, the expanded nanofiber structures can be sterilized using various methods (e.g., by treating with ethylene oxide gas, gamma irradiation, or 70% ethanol). In certain embodiments, the expanded nanofiber structure and/or nanofiber swab are sterilized by treating with ethylene oxide.

The instant application also encompasses the nanofiber swabs synthesized by the methods of the instant invention. Compositions comprising the nanofiber swabs synthesized by the methods of the instant invention and at least one pharmaceutically or biologically acceptable carrier are also encompassed by the instant invention.

In accordance with the instant invention, nanofiber swabs are provided. In certain embodiments, the nanofiber swab comprises an expanded, nanofiber structure, optionally, with a handle, wherein the expanded, nanofiber structure is attached to (e.g., covers and/or encompasses) the handle. In certain embodiments, the expanded, nanofiber structure of the nanofiber swab has an average diameter of about 3 mm to about 7 mm. In certain embodiments, the expanded, nanofiber structure of the nanofiber swab has an average length of about 10 mm to about 17 mm. In certain embodiments, the expanded, nanofiber structure is as described herein. In certain embodiments, the handle is as described herein.

In accordance with the instant invention, methods of collecting a sample (e.g., a biological sample) from or within a surface, object, or subject are provided. In certain embodiments, the method comprises contacting a nanofiber swab or expanded, nanofiber structure of the instant invention with a site in or on the surface, object, and/or subject. The site may be contacted more than one time with the nanofiber swab or expanded, nanofiber structure of the instant invention. The sample is collected from the site of the surface, object, and/or subject via the expanded nanofiber structure of the nanofiber swab. In certain embodiments, the sample comprises a cell. In certain embodiments, the sample comprises bacteria. In certain embodiments, the sample comprises a virus (e.g., a SARS-CoV-2 virus). In certain embodiments, the sample comprises a nucleic acid molecule (e.g., DNA, RNA, mRNA, etc.). In certain embodiments, the sample comprises a polypeptide, protein, or peptide. In certain embodiments, the site is within the subject's head (e.g., into a cavity (e.g., nasal cavity, nasopharynx cavity, or oropharynx). In certain embodiments, the site is contacted more than one time with the nanofiber swab. In certain embodiments, the contacting of the site comprises rotating and/or wiping the nanofiber swab on and/or around the site. In certain embodiments, the method further comprises placing the expanded nanofiber structure in a container (e.g., a tube). In certain embodiments, the container comprises a carrier. In certain embodiments, the carrier preserves the sample dislodged from the expanded nanofiber structure. In certain embodiments, the sample obtained from the surface, object, or subject is analyzed (e.g., identified).

In accordance with the instant invention, methods of cleaning a surface or object are provided. In certain embodiments, the method comprises contacting a nanofiber swab or expanded, nanofiber structure of the instant invention with a site in or on the surface or object. The site may be contacted more than one time with the nanofiber swab or expanded, nanofiber structure of the instant invention. In certain embodiments, the method removes a cell, bacteria, dust, and/or a virus from the site. In certain embodiments, the contacting of the site comprises rotating and/or wiping the nanofiber swab on and/or around the site. In certain embodiments, the method further comprises placing the expanded nanofiber structure in a container (e.g., a tube). In certain embodiments, the container comprises a carrier. In certain embodiments, the carrier preserves the sample dislodged from the expanded nanofiber structure. In certain embodiments, the sample obtained from the surface, object, or subject is analyzed (e.g., identified).

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In certain embodiments, hydrophobic polymers may have aqueous solubility less than about 1% wt. at 37° C. In certain embodiments, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

As used herein, the term “subject” refers to an animal, particularly a mammal, particularly a human.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following example illustrates certain embodiments of the invention. It is not intended to limit the invention in any way.

### Example

### Materials and Methods

### Materials

PCL (Mw=80 kDa), gelatin (porcine), Pluronic® F-127, and NaBH₄ were all purchased from Sigma-Aldrich (St. Louis, Mo.). Dichloromethane (DCM) and N, N-dimethylformamide (DMF) were purchased from Oakwood Chemical (Estill, S.C.). Plastic sticks were purchased from Amscan Inc. (Elmsford, N.Y.) and Z-poxy was purchased from Pacer Technology, Inc. (Ontario, CA). Cotton swabs (6 in, Cotton-Tipped Applicators STERILE, wood shaft) were purchased from McKesson (Irvington, Tex.) and flocked swabs (6 in, PurFlock ULTRA®, Sterile Flocked Collection Devices) were purchased from Puritan Medical Products (Pittsfield, Me.). Dye (Assorted Food Color & Egg Dye) was purchased from McCormick & Company (Baltimore, Md.). Dulbecco's modified eagle medium (DMEM), fetal bovine serum (FBS), penicillin-streptomycin (PenStrep) were all purchased from Invitrogen (Carlsbad, Calif.). HEK293 cells were purchased from ATCC (Manassas, Va.). Blood was collected from sacrificed Sprague Dawley rats, which were purchased from Charles River Laboratories (Wilmington, Mass.). DNA quantification kits were QIAamp® DNA Investigator Kit which was purchased from Qiagen (Venlo, Netherlands). MRSA USA300 LAC was obtained in-house at the University of Nebraska Medical Center (UNMC). Columbia CAN with 5% sheep blood agar medium was purchased from Remel (Lenexa, Kans.) and tryptic soy broth (TSB) bacterial medium was purchased from Thermo Fisher Scientific Inc. (Waltham, Mass.). SARS-CoV-2 (Strain: BEI_USA-WA1/2020) was obtained from BEI Resources. Phosphate Buffered Saline (PBS) was purchased from Fisher Scientific (Hampton, N.H.). QIAamp® Viral RNA Mini Kit was purchased from Qiagen (Venlo, Netherlands). RT-PCR (2X) Master Mix and SuperScript™ III Platinum Taq Mix was purchased from Invitrogen (Carlsbad, Calif.). Primers and Probe for RT-PCR was purchased from Integrated DNA Technologies (Coralville, Iowa). QuantStudio™ 3 instrument and software was purchased from Applied Biosystems (Foster City, Calif.). A vacuum (Black & Decker Dustbuster®) for vacuumed mass loss was purchased from Black & Decker (New Britain, Conn.).

### Fabrication of PCL Nanofiber Mats

PCL nanofiber mats were generated via electrospinning (Chen, et al., Appl. Phys. Rev. (2020) 7(2):021406; Chen, et al., Nano Lett. (2019) 19(3):2059-2065; Chen, et al., Biomaterials (2018) 179:46-59; Chen, et al., Acta Biomater. (2020) 108:153-167; Chen, et al., Adv. Mater. (2020) 32(43):2003754). PCL pellets (10% w/v) and Pluronic® F-127 (0.5% w/v) were dissolved in a 4:1 (v/v) DCM:DMF solution. The resulting PCL/F-127 solution was electrospun at 16 ml/hour at 25° C., 55% RH, and under 32 kV applied voltage using an LE-100 electrospinning machine with a circular 20 emitter array equipped with 21 gauge needles (such that each needle had a flow rate of 0.8 ml/hour). The nanofibers were collected on a high-speed drum collector until an approximately 1 mm thick mat was obtained.

### Fabrication of Swab Tips

The nanofiber mat was removed from the drum and cut into different sized rectangles while submerged in liquid nitrogen, with the width of the rectangle serving as the radius of the swab. In this case, for each study, rectangles were cut measuring roughly 2.5 mm wide by 13.5 mm length. Mats were completely soaked in the liquid nitrogen before cutting to ensure no fusion could occur between fibers. Next, rectangles were removed from the liquid nitrogen and thermally fused along one of the long sides (applying heat perpendicular to the face of the rectangle) by melting the edge on an 85° C. hot plate. Once the rectangles cooled, they were added to a 1 M NaBH₄ solution and left overnight to expand into cylinders. After expansion, the cylinders were washed with distilled water three times expanded under vacuum at 200 Pa three times. After the third vacuum, all water was removed and the cylinders were freeze-dried. Once dry, swab tips were removed and submerged in a 0.75% gelatin solution and allowed to soak for 20 minutes. Gelatin-coated swab tips were directly freeze-dried again. Once dry, swab tips were separated and crosslinked in a glutaraldehyde (GA) chamber for 24 hours. After crosslinking, plastic sticks were inserted into the middle of each cylinder and bound with epoxy. Finally, before use, each swab was sterilized with ethylene oxide (EtO).

### Characterization of Swabs

Each swab was imaged using a camera and scanning electron microscope (SEM) (FEI, Quanta 200, Oregon). SEM images of each swab were taken in sections and combined using Adobe Photoshop's merge function. Merged edges were smoothed and the composite image was set on a black background. SEM images were taken at 200× and 1556×. The diameters of each swab fibers were measured using ImageJ. The dimensions of each swab were measured using a digital caliper (Mitutoyo, Kanagawa, Japan). Porosity of each nanofiber was estimated based on the volume difference between the whole swab and the raw materials of the swab, which is summarized in the following equation:

η=(Vₛₜ−V_{bm})/Vₛₜ×100% (1)

Where η is porosity, Vₛₜ the volume occupied by the swab tip, and V_{bm} is the volume occupied by the bulk material comprising the swab tip. Bulk volume of each swab is assumed to be V_{bm}=m_{bm}/ρ_{bm}, where m_{bm} is the mass of the bulk swab tip material, and ρ_{bm} is the density of the bulk swab tip material. Both flocked and nanofiber swabs were assumed cylindrical, while cotton swabs were assumed egg-shaped in generating Vₛₜ. Density values were assigned based on the listed material composition of each swab tip. A summary of measurements and material values are provided in Table 1.

TABLE 1
Swab measurements and material properties.

| parameter | cotton | flocked | nanofiber |
|---|---|---|---|
| mₛₜ (g) | 0.0533 | 0.0527 | 0.00840 |
| dₛₜ (mm) | 5.01 | 5.00 | 5.18 |
| lₛₜ (mm) | 13.0 | 22.4 | 13.6 |
| vₛₜ (mm³) | 113.205 | 339.077 | 265.302 |
| v_{bm} (mm³) | 34.167 | 45.826 | 7.336 |
| ρ_{bm} (g/mm³) | 0.00156 | 0.00115 | 0.00115 |
| ηₛₜ (%) | 69.82 | 86.49 | 97.23 |

ₛₜ= swab tip.
_{bm}= bulk material.

### Mass Loss

In order to ascertain inhalation of nanofibers from nanofiber swabs did not pose as a risk, two different mass change studies were carried out. First, initial swab masses were taken. Next, swabs were placed directly into a small opening in a handheld Black & Decker Dustbuster® vacuum and suctioned at approximately 18 airwatts for 5 seconds each and final volume was recorded. Mass change was expressed as a mass change percent of the initial swab mass. Additionally, each swab type was subjected to 50 and 100 abrasive cycles with twists. One abrasive cycle was considered as one full back-and-forth swab on a flat surface. Masses were taken before abrasion, after 50 cycles, and after 100 cycles, and percent mass change was expressed as a percent of the initial swab mass.

### Dye Absorption and Release

Blue dye was added at a 1:10 ratio to distilled water to create a stock dye solution. Six serial 10× dilutions were performed and absorbance measured at 620 nm using a Synergy H1 Hybrid MicroPlate Reader (BioTek, Winooski, Vt.) to create a standard curve using a final volume of 2 ml. Next, initial swab masses were taken, swabs were immersed in stock dye solution, and a final weight was taken. Absorption ratio was calculated as:

Absorption Ratio=(m_{f}−mᵢ)/mᵢ×100% (2)

Each swab was submerged in 100×diluted dye solution for 10 seconds, removed, and tip placed into a 5 ml conical tube with 2 ml of water and vortexed for 10 seconds. The suspension was plated and measured on the microplate reader at 620 nm. To determine the amount of released dye, the maximum absorbable volume for each swab tip was determined with cotton absorbing the smallest volume near 120 μl. Subsequently, 50 μl of the 100× diluted dye solution was pipetted directly onto the swab tips (experimental) or directly into 2 ml water (standard). The tips were then placed in 5 ml tubes with 2 ml of distilled water, vortexed for 10 seconds, and absorbance measured on the microplate reader. The released dye was expressed as:

Released Dye=abs_{sw}/abs_{std}×100% (3)

Where, abs_{sw} is the absorbance measured from the dye solution released from the swab tips and abs_{std} is the absorbance measured from the 50 μl of dye solution, both of which were diluted into 2 ml of water.

### Protein Swabbing

Stock Bovine Serum Albumin (Pierce BCA Protein Assay Kit, Pierce, Rockford, Ill.) was used as a model protein for both surface and solution swabbing. During wet extraction (solution swabbing), each swab tip was immersed in a 1500 μg/μl solution of BSA and distilled water for 10 seconds with gentle stirring. After removal, swab tips were added to 5 ml conical tubes with 2 ml of working reagents and incubated following the BCA Protein Assay Kit protocol. Absorbance values were measured using a microplate reader and compared to a standard curve generated from known concentrations of BSA protein. The extraction rate was measured as the ratio of total protein detected after swabbing:total protein in solution. To measure surface protein recovery, 500 μl of 1000 μg/μl BSA solution was pipetted onto a glass slide and allowed to dry. Swab tips were moistened by dipping in distilled water and the dried BSA was directly swabbed. Swab tips were added to a 5 ml conical tube with 2 ml working reagent, incubated, and analyzed with a microplate reader. Recovered protein was measured as the ratio of detected protein from the swabs to the known protein amount in the dried solution.

### Cell Swabbing

Human embryonic kidney cells (HEK 293) were cultured in completed DMEM+10% FBS+1% PenStrep until confluent. After reaching confluence, cells were trypsinized and resuspended in fresh media which was subsequently aliquoted into individual 50 ml tubes. Each swab was submerged in the cell suspension, moving back and forth for 10 seconds. After removal, the tips of each swab were added directly to 10 ml of fresh media. A small sample from each resuspension was stained with trypan blue and counted using a hemocytometer, from which the total cell count was estimated. Similarly, HEK 293 cells were grown to confluence on a petri dish. Media was removed from each petri dish and the cell monolayer was directly swab using each swab type. The tips of each swab were added directly to 10 ml of fresh media and vortexed for 10 seconds. Resuspended cells were stained with trypan blue and counted using a hemocytometer, from which the total cell count was estimated. Small volumes of cell suspension from the solution and surface swabbing were imaged using a field microscope.

### Blood Swabbing and DNA Detection

Fresh blood was collected from Sprague Dawley rats and kept on ice until ready for use. Known volumes of blood were added to 2 ml microcentrifuge tubes and allowed to dry completely. DNA was isolated using a Qiagen DNA Investigator Kit and quantified using a NanoDrop™ OneC Spectrophotometer (Thermo Fisher Scientific, Waltham). Next, 25 μl, 50 μl, and 100 μl of blood was pipetted onto a glass surface and allowed to dry. Swab tips were moistened in distilled water and dried blood was directly swabbed. Swab tips were vortexed in buffer for 10 seconds and DNA isolation and quantification was carried out as described. Additionally, blood absorption was also measured by weighing dry swab mass, immersing the swab tips in blood (35° C.) for 10 seconds, and measuring the final mass of the swabs with absorbed blood. The equation used to generate blood absorption percent is given in Equation 2. Released DNA was measured by pipetting 50 μl of blood directly onto each swab tip, isolating and measuring the DNA, and expressing the release as the ratio of detected DNA from the swabs to total DNA from 50 μl of blood.

### SARS-CoV-2 Swabbing and Detection

An initial stock of SARS-CoV-2 with a titer of 1.30×10⁵ plaque forming units per ml (pfu/ml) was serially diluted in PBS by single log dilution down to 1.30×100 pfu/ml. A copy of each dilution was created for each swab type in 50 ml conical tubes for a total of 3 tubes per dilution. Each swab was dipped into its dilution tube and pressed against the insides of its respective 1.5 ml microcentrifuge tube already containing 500 μl of PBS for sample collection. Each 1.5 ml microcentrifuge tube was vortexed for 10 seconds to ensure thorough sample mixture into the PBS. The sample collection was conducted in triplicates for each swab type and dilution. A negative control for each swab type consisted of the 50 ml conical tube containing PBS only while repeating the same sample collection method. 140 μl of each collected sample was taken for RNA isolation via Qiagen QIAamp® Viral RNA Mini Kit. The viral RNA was analyzed by RT-PCR. The RT-PCR was performed on the QuantStudio™ 3. The RT-PCR reactions underwent an initial condition of 55° C. for 10 minutes then a 94° C. for 4 minutes followed by 45 cycles of 94° C. for 15 seconds and 58° C. for 30 seconds. The RT-PCR blank sample control resulted in no amplification. Each RT-PCR reaction consisted of 5.6 μl nuclease free water, 12.5 μl Invitrogen (2×) Master Mix, 0.4 μl MgSO₄, 1.0 μl Primer/Probe Mix (0.15 μl Forward Primer (100 μM stock), 0.2 μl Reverse Primer (100 μM stock), 0.05 ηl Probe (100 μM stock), 0.6 μl TE Buffer), 0.5 μl SuperScript™ III Platinum Taq Mix, and 5.0 μl extracted Sample RNA. *E gene target primers and probe:

Probe:
(SEQ ID NO: 1)
5′/56-FAM/ ACACTAAGCCATCCTTACTGCGCTTCG/3AIBKFG/-3′
Forward Primer:
(SEQ ID NO: 2)
5′-ATATTGCAGCAGTACGCACACA-3′
Reverse Primer:
(SEQ ID NO: 3)
5′-ACAGGTACGTTAATAGTTAATAGCGT-3′

### Statistics and Graphics

All experiments and measurements were done with a minimum of three replicates. Data is expressed in tables and graphs as the mean±standard deviation. All physical measurements were taken on a calibrated digital caliper and all photographic measurements were made using ImageJ. Ordinary one-way ANOVAs, paired and unpaired t-tests, two-way ANOVAs (displaying significance within-group), and frequency distributions were used where appropriate. Post-hoc testing (Tukey's), residual distribution, and normality were checked following each analysis. Definitive outliers were identified and removed using Iterative Grubbs' testing. Interpretation of the SARS-CoV-2 data included within group comparisons except at 10¹ and 10⁰ pfu/ml, where no virus was detected. Each data set was analyzed and charted using GraphPad Prism Version 9.0.0. Significance was denoted as ns≥0.05, 0.01<*p<0.05, 0.01<** p<0.001, 0.001<***p<0.0001, ****p<0.0001. All graphics are original and were prepared using BioRender.

### Results

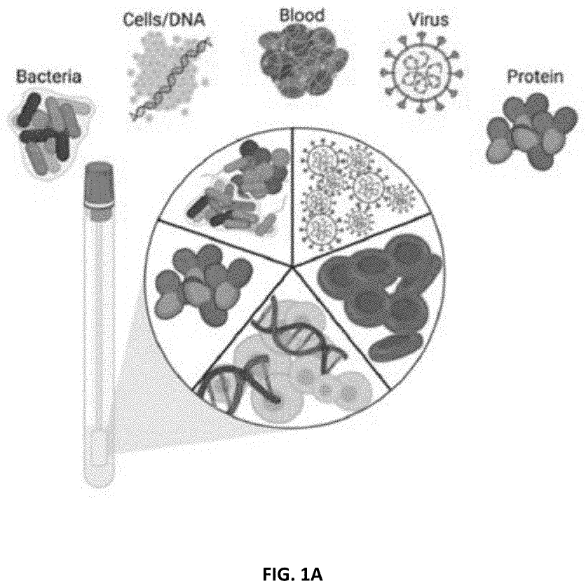

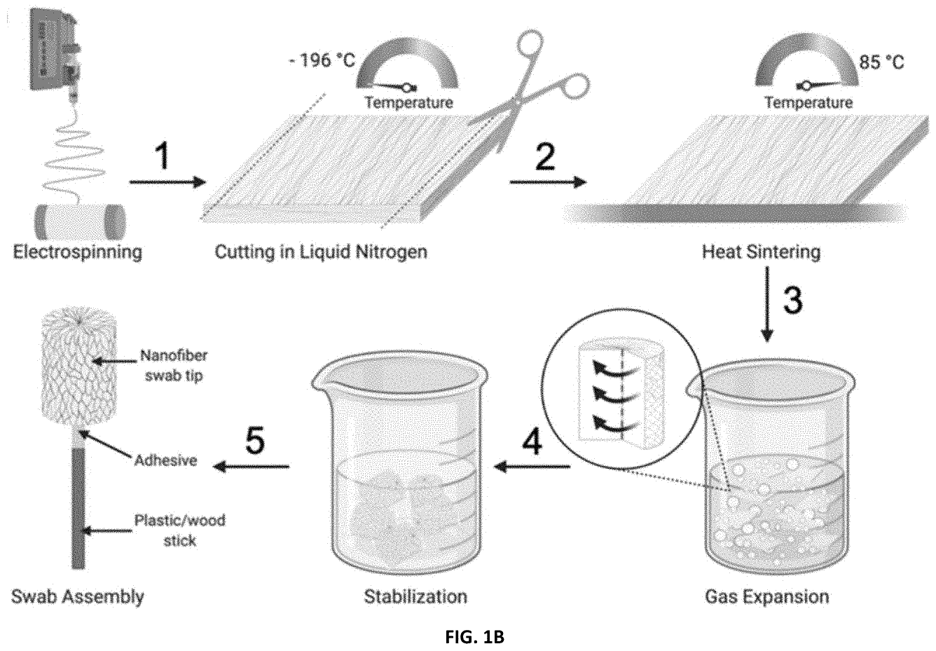

Superabsorptive 3D nanofiber matrices with layered structures can be synthesized by expanding electrospun nanofiber membranes using an innovative gas foaming technique (Chen, et al., Biomaterials (2018) 179:46-59). By applying the solids-of-revolution concept to the gas foaming, various nanofiber objects (e.g., spheres, cylinders, and cones) can be generated from expanding electrospun nanofiber membranes around a thermally fixed axis (Chen, et al., Nano Lett. (2019) 19:2059-2065). Herein, novel nanofiber swabs were synthesized that improve sample collection and release and test sensitivity in a variety of applications. More specifically, radially aligned, 0.75% w/v gelatin-coated PCL nanofiber objects with cylindrical shape were generated and then bonded to plastic swab sticks. The performance of the novel nanofiber swabs was compared in analyzing different biological materials including bovine serum albumin (BSA), HEK293 cells, DNA from blood, Methicillin-resistant Staphylococcus aureus (MRSA), and SARS-CoV-2 (FIGS. 1A, 1B) to two other swab types, cotton and flocked.

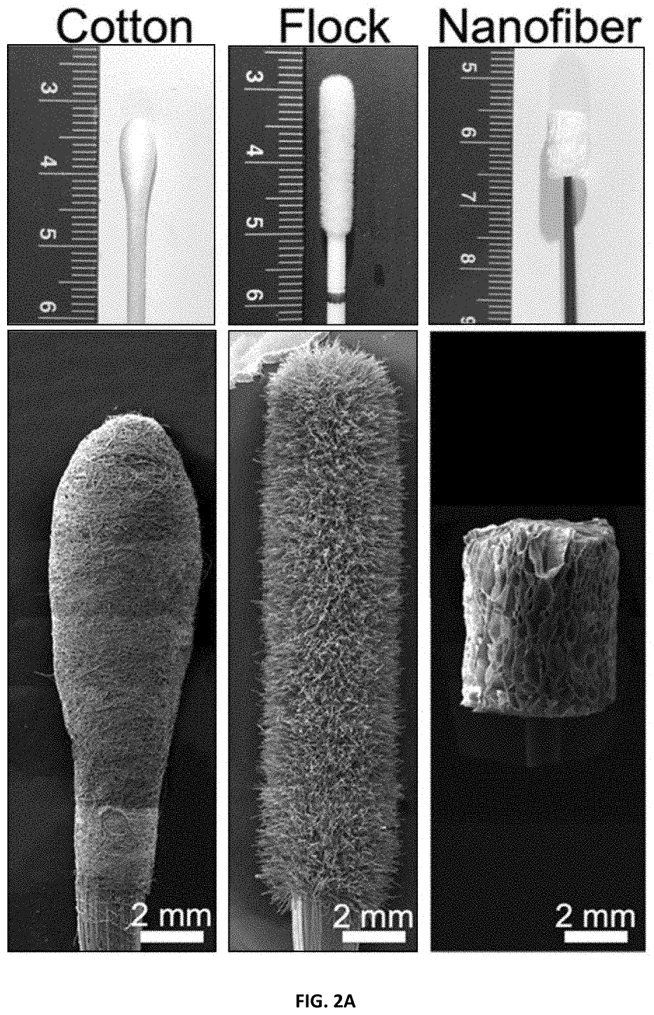

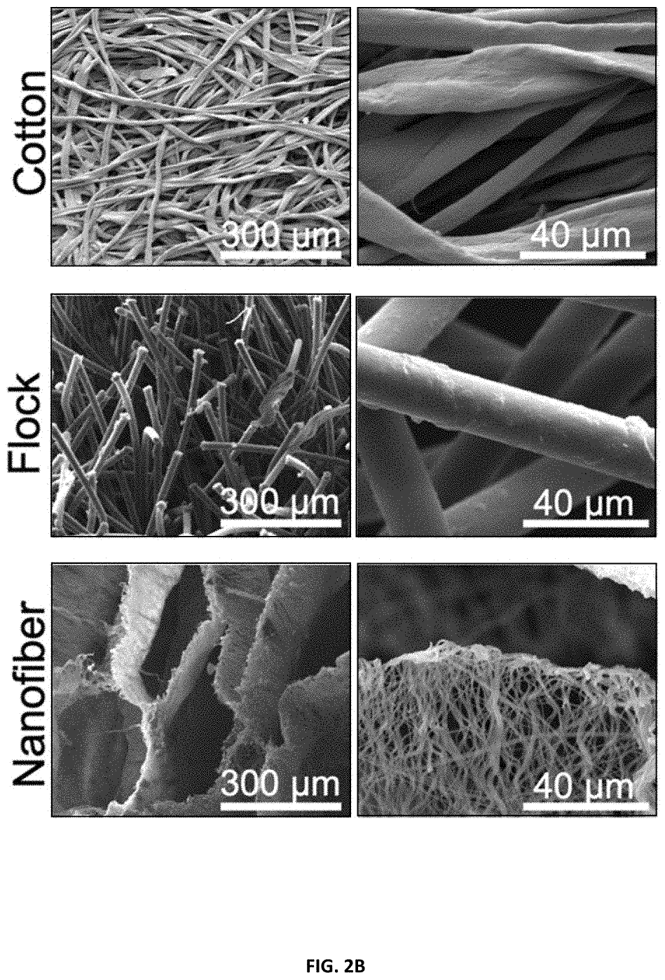

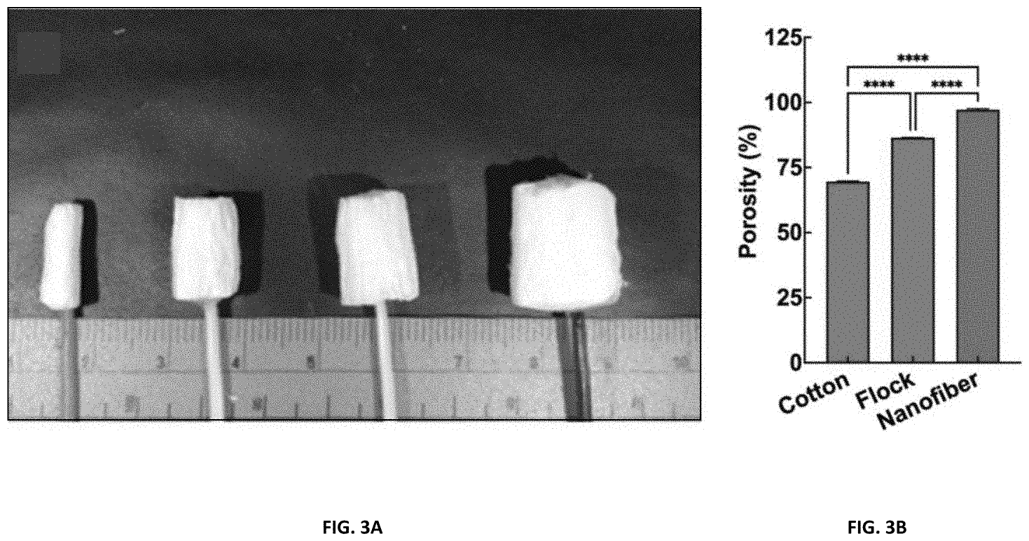

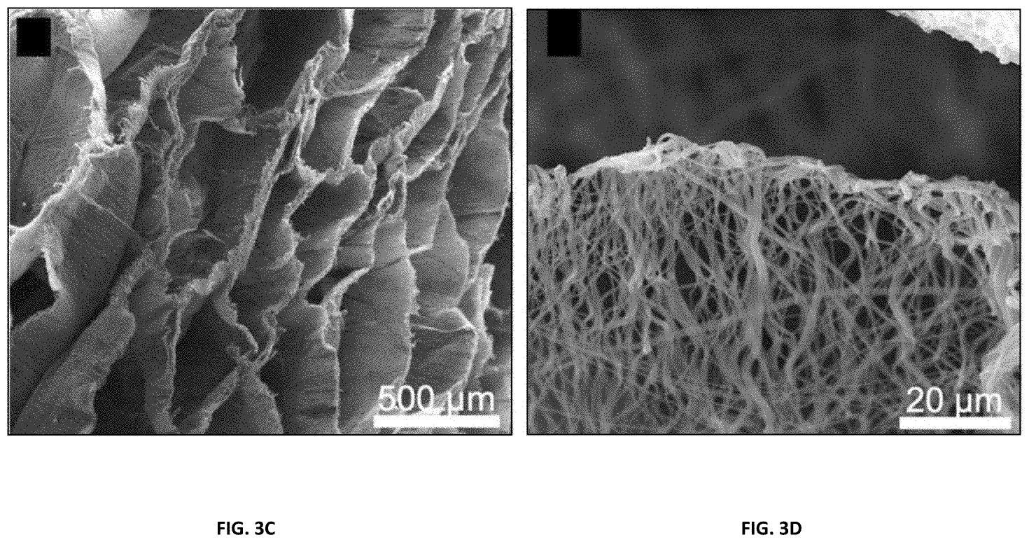

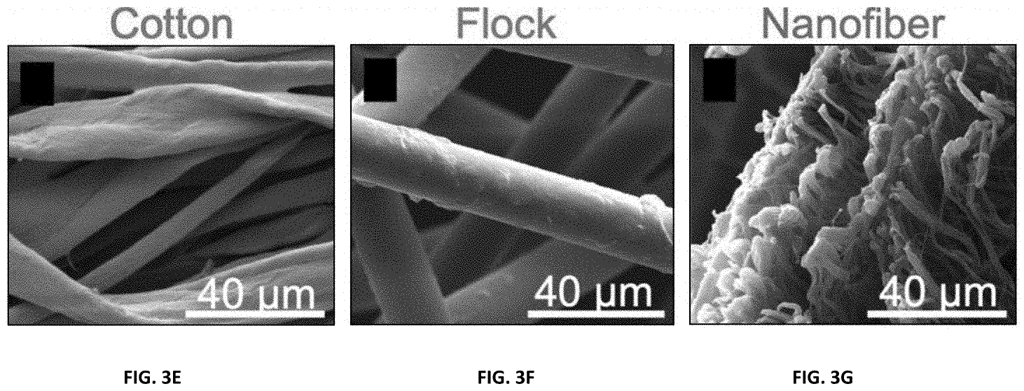

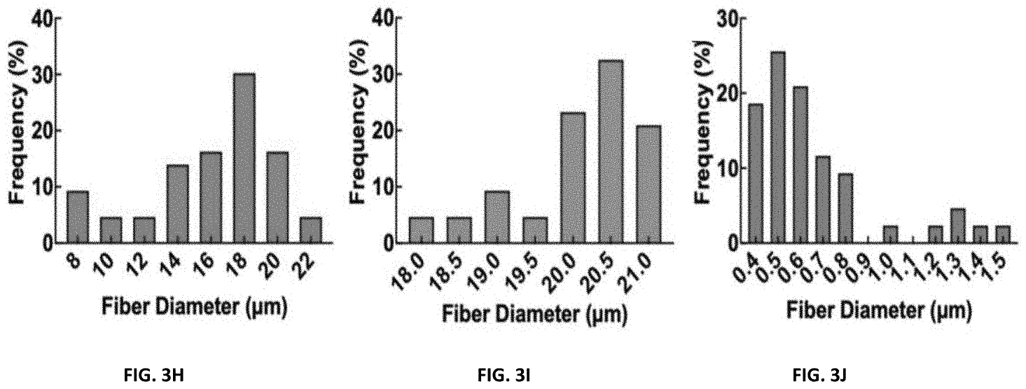

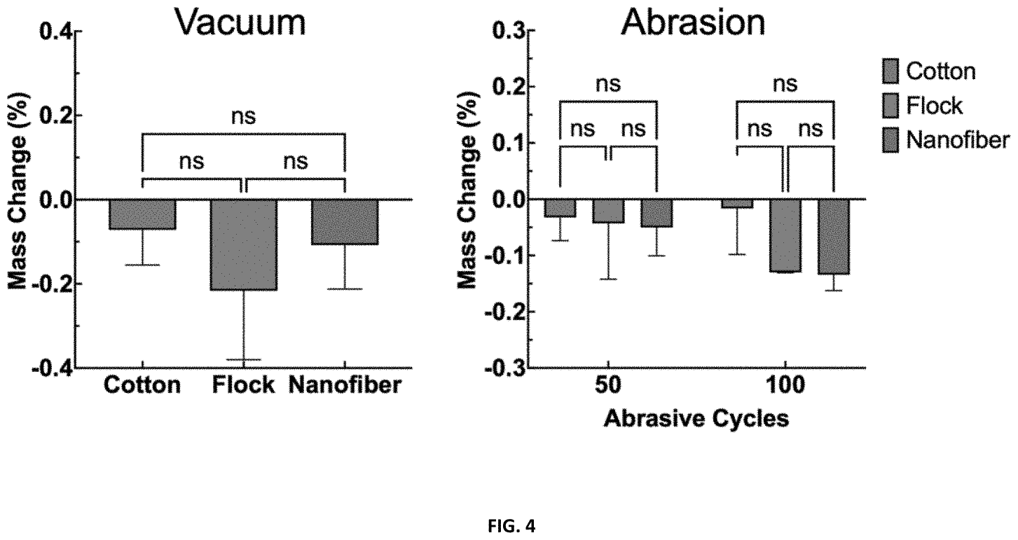

Ultimately, swab performance is directly related to their morphology. The three swab types including cotton, flocked, and nanofiber were characterized and compared. Cotton swabs (sterile 6 in, McKesson) have approximately 1 cm long ovular tips while flocked swabs have roughly 2 cm long cylindrical tips. Nanofiber swabs used in each experiment were cylindrical and approximately 1 cm long (FIG. 2A). SEM images reveal that cotton swabs consist of layered microfibers (˜18 m diameter) and therefore have a relatively low surface-area-tovolume ratio (FIG. 2B, FIG. 3E, 3J). In contrast, flocked swabs (sterile 6 in, Puritan) appear as vertically aligned microfibers (˜20 m diameter, ˜500 m length) and seemingly have a much higher surface-area-to-volume ratio, as is characteristic of flocked objects (FIG. 2b, FIG. 3F, 3I) (Hitzbleck, et al., Adv. Mater. (2013) 25:2672-2676; Gossla, et al., Acta Biomater. (2016) 44:267-276). Nanofiber swabs showed hierarchical structures with large primary pores between expanded fiber layers and smaller secondary pores visible within each fiber layer with nanofiber diameters of approximately 500 nm (FIG. 2B, FIG. 3C, 3D, 3G, 3J). Nanofiber swabs were significantly more porous than both flocked and cotton swabs despite having relatively similar volumes and bulk material densities (FIG. 3B, Table 1). Further, nanofiber swabs can be prepared at various sizes without changing their porous features or nanofibrous morphology, thus allowing for size tailoring based on predesignated applications (FIG. 3A). Ensuring stability and abrasion resistance of the nanofiber swabs was crucial, as inhalation of nanoparticles can cause respiratory damage (Byrne, et al., Mcgill J. Med. (2008) 11:43-50). To ensure the 0.75% gelatin coating supplied sufficient abrasion resistance and stability, mass change of each swab was determined after vacuuming and abrasive cycles (to mimic possible mechanical abrasion and sharp inhalation during nasopharyngeal swabbing). No significant mass loss was detected after substantial vacuuming or abrasive cycling in any swab type (FIG. 4).

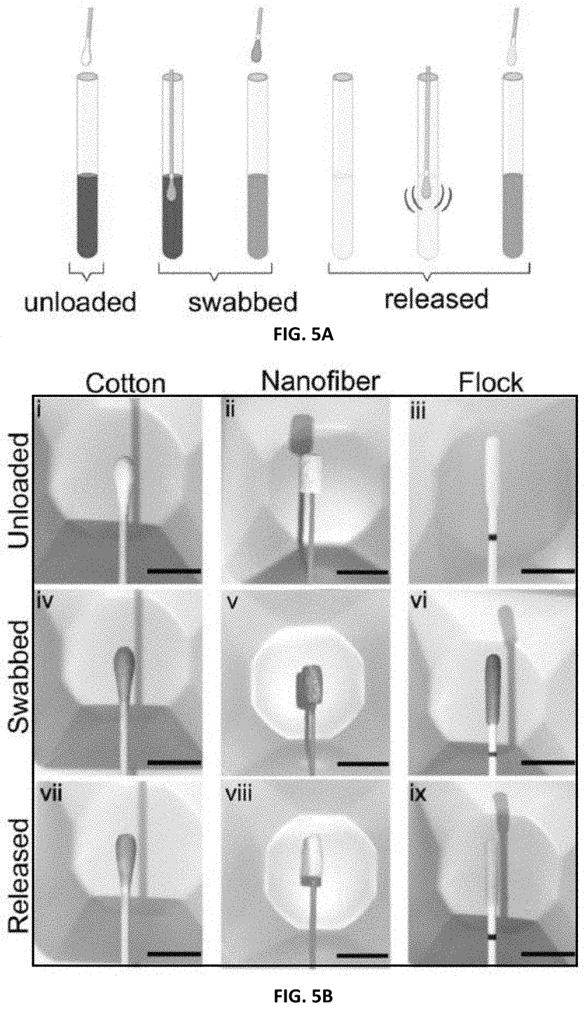

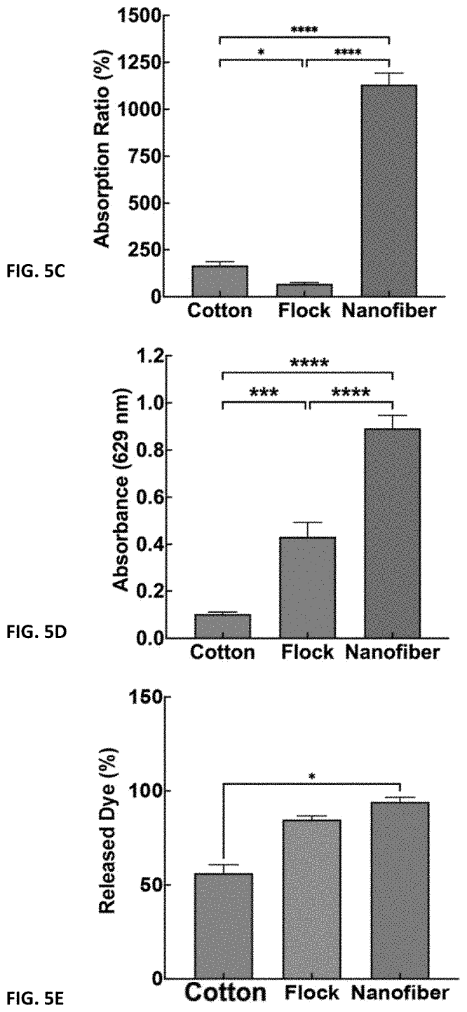

To examine the collecting efficacy, blue dye solutions (water with blue food coloring (McCormick & Company, Md.)) were swabbed and colorimetric changes were compared (FIG. 5A). All swab types appeared dark after swabbing the solution, and flocked and nanofiber swabs appeared to return almost back to white after releasing the dye (FIG. 5B). Arguably the most critical performance indicator for swabs, absorption was tested by measuring absorption ratio (% of initial weight) after submersion in water. Nanofiber swabs demonstrated outstanding absorption relative to flocked and cotton swabs, absorbing over 1100% of its dry mass (FIG. 5C). The raw absorbance (measured at 629 nm) of the solution into which dye was released by each swab was measured. Assumedly due to the large volume of dye solutions the nanofiber swabs absorbed, the absorbance value was significantly higher for the nanofiber swabs compared to both flocked and cotton swabs. However, flocked swabs also achieved significantly higher absorbance compared to cotton swabs despite absorbing less (FIG. 5D). To determine the contribution release had on overall absorbance, 50 μl of dye solution was pipetted onto each swab tip and directly into the release solution (serving as the control). Release was quantified as the ratio of swab absorbance to control. Nanofiber and flocked swabs released more than 80% of the 50 μl and did not significantly vary, although both significantly outperformed cotton, which released slightly over 50% (FIG. 5E). Though simple, this experiment demonstrated that flocked and nanofiber swabs released the majority of the dye, but that nanofiber swabs absorbed significantly more, giving them an overall superior performance.

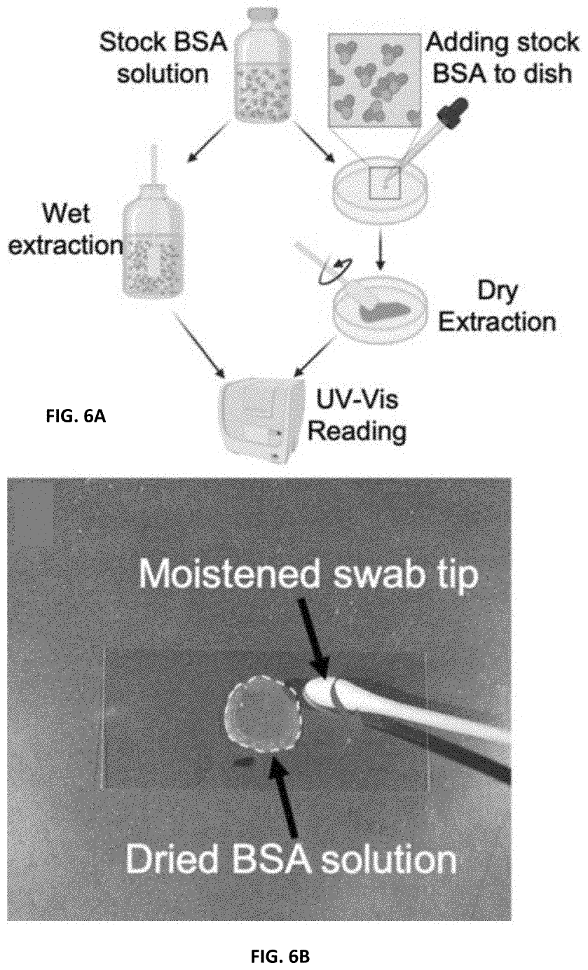

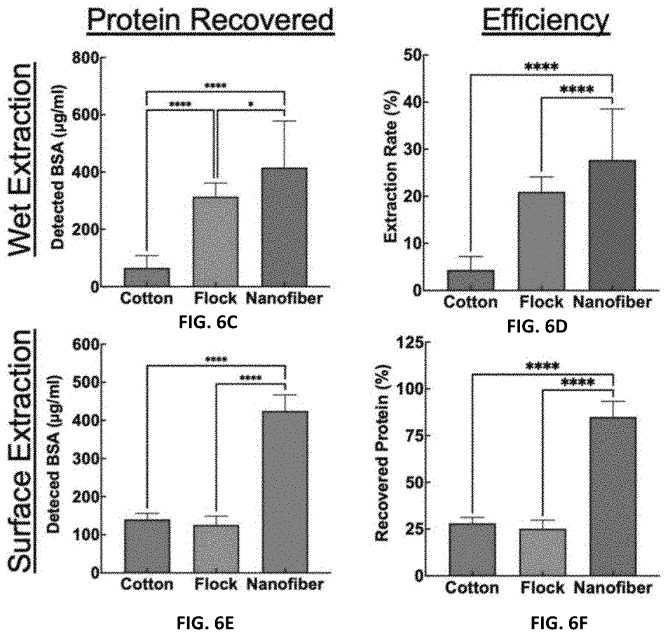

Detecting proteins from surfaces and wet samples is commonplace in food safety and medical diagnostics (Lahou, et al., Int. J. Environ. Res. Public Health (2014) 11:804-814; Keeratipibul, et al., Food Control (2017) 77:139-144; Topkas, et al., Clin. Chim. Acta (2012) 413:1066-1070). To evaluate and compare the efficacy of nanofiber swabs at detecting protein from surfaces and solutions, BSA was used as a model protein. A BSA solution of a known concentration was either directly swabbed (wet extraction) or a known volume was added to a glass slide and allowed to dry before swabbing with a premoistened swab (dry extraction) (FIG. 6A, 6B). After swabbing, each swab tip was vortexed in the Micro BCA protein assay working reagent and analyzed using a UV spectrophotometer. Absorbance values were correlated to protein content (μg/μl) using a set of standards. Known BSA concentrations were used to determine the extraction rate (the percent of detected solution swabbed BSA/detected standard BSA×100) and recovered protein (the percent of detected surface swabbed BSA/detected standard BSA×100). During wet extraction, nanofiber swabs detected significantly more protein than both flocked and cotton swabs, though flocked swabs also showed significantly higher protein detection than cotton alone. The higher absorptive properties of the nanofiber swabs allow them to outperform cotton and flocked swabs, releasing more than 25% of the protein from the given solution (FIG. 6C, 6D). Similarly, nanofiber swabs outperformed flocked and cotton swabs during surface extraction, detecting nearly 4 times as much protein, which was more than 75% of the protein from the dried sample (FIG. 6E, 6F). Flocked swabs are excellent at releasing biological specimens, and several studies indicate that flocked swabs are adequate or superior for surface detection (Brownlow, et al., J. Forensic Sci. (2012) 57:713-717; Probst, et al., Appl. Environ. Microbiol. (2010) 76:5148-5158; Dalmaso, et al., PDA J. Pharm. Sci. Technol. (2008) 62:191-199). Here, it is demonstrated that nanofiber swabs outperformed flocked swabs in wet and dry protein detection. Mechanistically, nanofiber swabs detect more protein from solution due to the high absorptivity of the swabs. During dry extraction, the same volume of solution was added to each swab tip, indicating that the nanofiber swabs removed more protein from the surface mechanically. Morphologically, nanofiber swabs contact the sample surface with more contact area, given the nature of their nanofibrous composition. Additionally, the rigidity and plasticity of the nanofiber swabs allow them to be slightly more abrasive and thus remove samples from surfaces more easily.

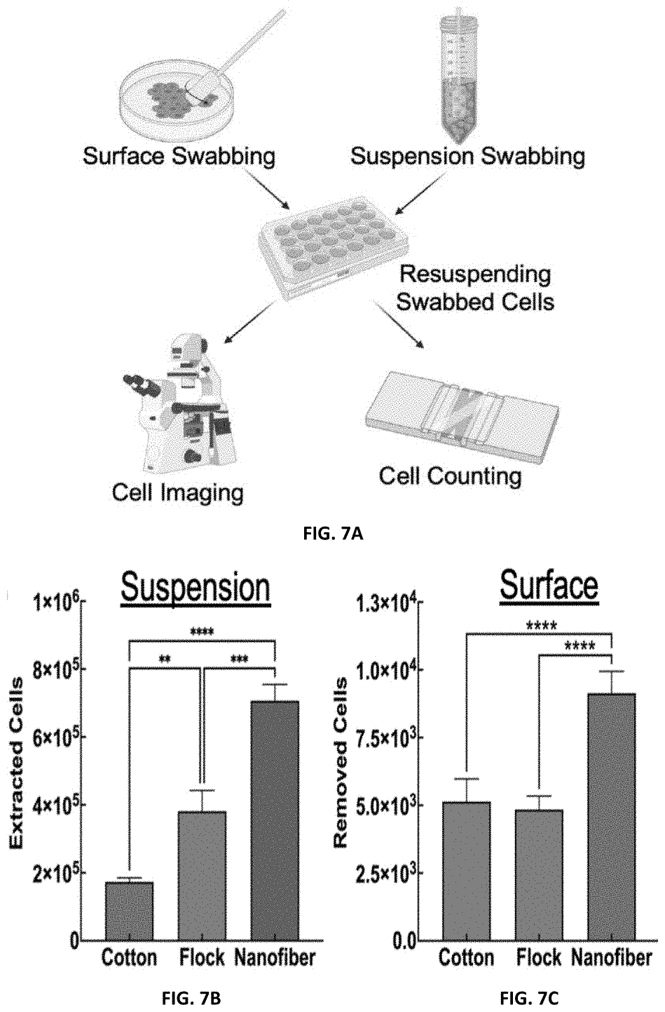

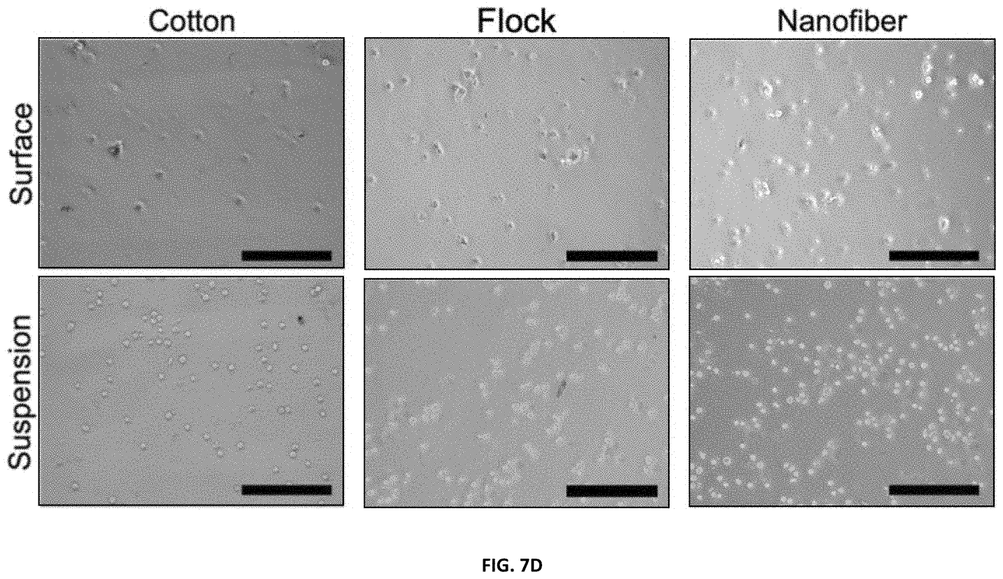

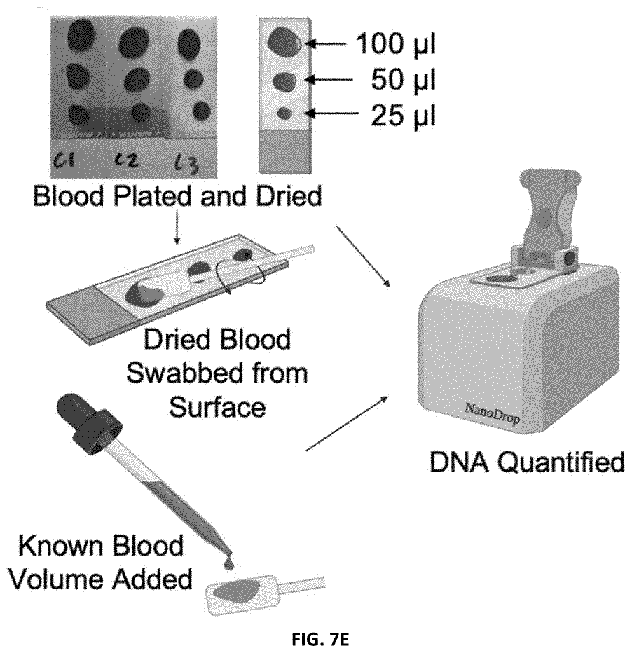

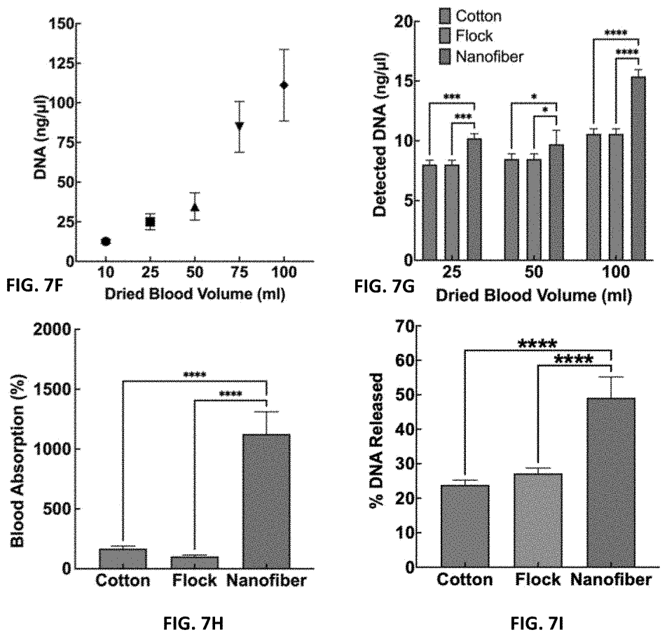

Next, the swabs were used to both extract HEK293 cells from a suspension and physically remove them from a plate-adhered tissue monolayer (FIG. 7A). During suspension swabbing, HEK293 cells were suspended at a known concentration in media and directly swabbed with each swab. Surface swabbing was performed on a confluent cell layer cultured in a Petri dish. For both suspension and surface swabbing, tips were vortexed in fresh media and resuspended cells were counted using hemocytometry and imaged with a field microscope. In both applications, nanofiber swabs showed significantly higher cell counts compared to flocked and cotton swabs (FIG. 7B, 7C). In the suspension study, the driving mechanism for superior nanofiber performance is the super-absorptivity and high degree of release. Interestingly, nanofiber swabs also had the highest yield during surface swabbing, which indicates that the plasticity of the swabs is sufficient for use as a surface-detecting swab. Notably, flocked swabs outperformed cotton swabs in suspension but not in surface swabbing, due to the low plasticity of the flocked fibers and their subsequent inability to remove cells from a monolayer due to poor abrasion. Microscopy imaging confirmed the relative performance of each swab type and also revealed possible contaminants/debris, although the amount of debris appeared consistent with each swab type (FIG. 7D).

Swabs are heavily used in forensic science for DNA extraction from surfaces and aqueous solutions and are often used for patient, perpetrator, or victim identification, making them a vital tool in forensics (Schulz, et al., Forensic Sci. Int. (2002) 127: 128-130; Turingan, et al., Int. J. Legal Med. (2020) 134: 863-872; Kitayama, et al., Leg. Med. (2020) 46: 101713; Katilius, et al., Forensic Sci. Int.: Genet. (2018) 35: 9-13). To investigate the potential of nanofiber swabs in harvesting DNA, known volumes of blood were pipetted onto glass slides and allowed to dry completely. Premoistened swabs were used to extract DNA from the dried blood and analyzed using NanoDrop (FIG. 7E). Known volumes of blood were directly analyzed, showing a linearly increasing relationship between blood volume and detected DNA (FIG. 7F). All three swab types demonstrated the same linearly increasing relationship after surface swabbing dried blood, though nanofiber swabs detected significantly more DNA at each known blood volume compared to both cotton and flocked swabs (FIG. 7G). It is likely that the swabs reach a maximum absorptivity at a given volume of blood and therefore experience lower DNA yields at higher concentrations. To validate this, the blood absorption ratio of each swab type was determined, which shows that nanofiber swabs absorbed nearly ten times more than cotton and flocked swabs (FIG. 7H). Finally, DNA release efficiency was evaluated by pipetting 50 μl of blood directly onto each swab tip (noting that 50 μl was well below each swab tip's maximum absorption volume) and releasing in the DNA buffering solution. Nanofiber swabs exhibited nearly double the percent of released DNA compared to cotton and flocked swabs, once again demonstrating superior absorption and release of biological material. Improved swab sensitivity can help to identify persons of interest from smaller biological samples, potentially dramatically improving progress in certain forensic cases.

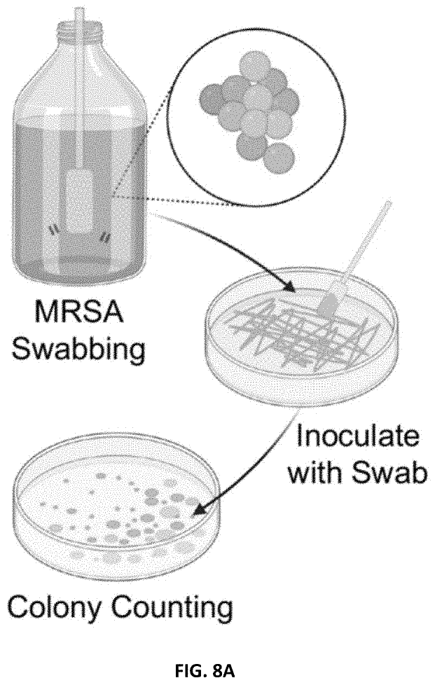

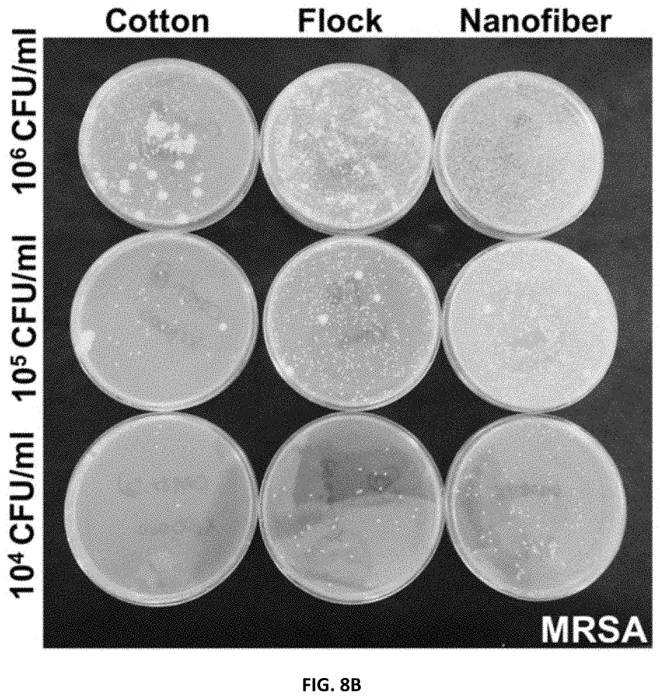

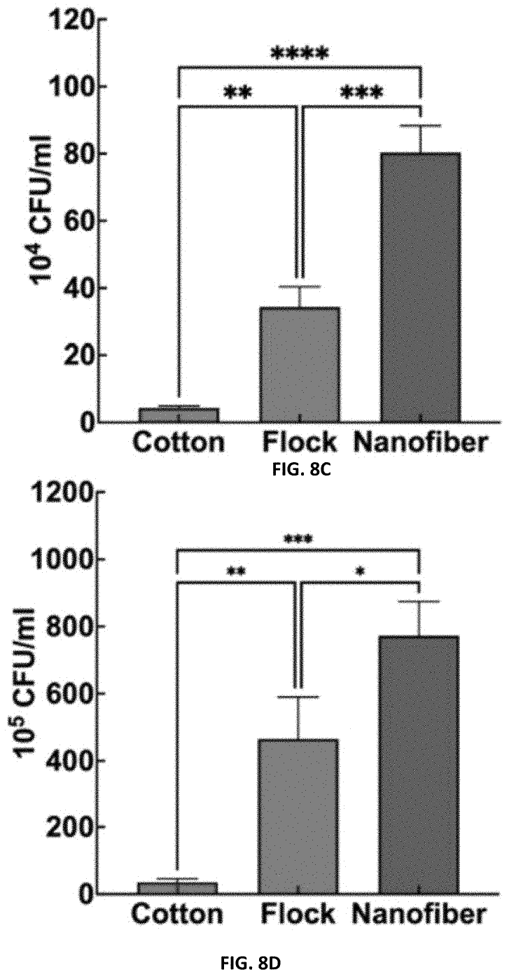

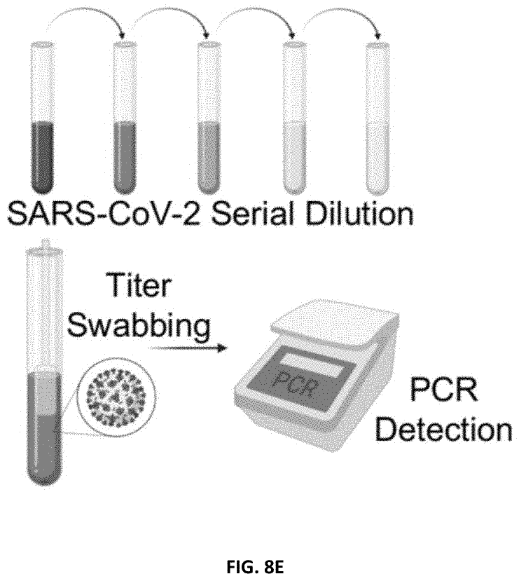

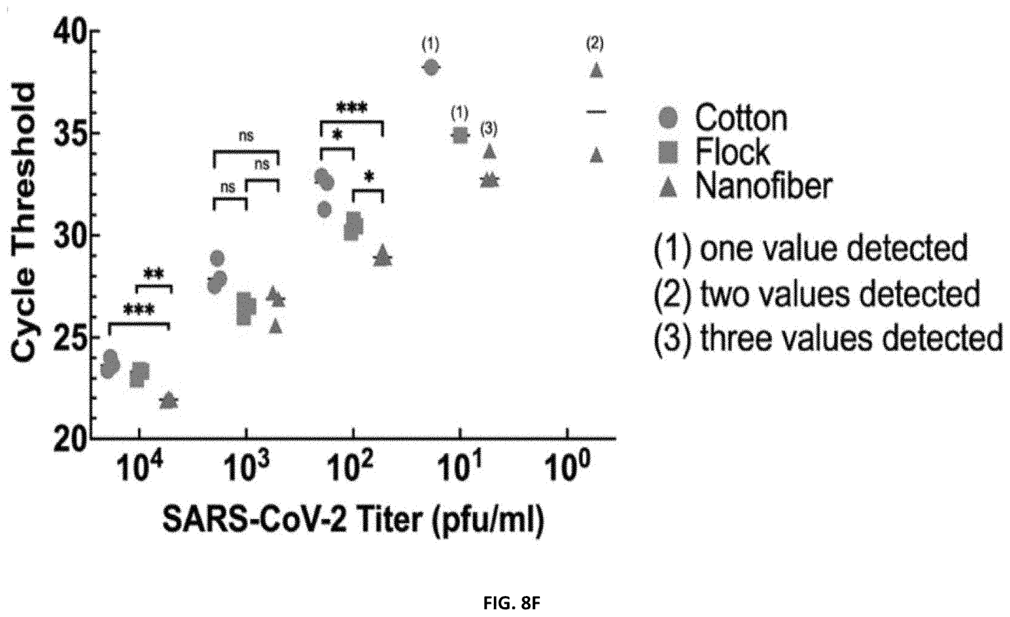

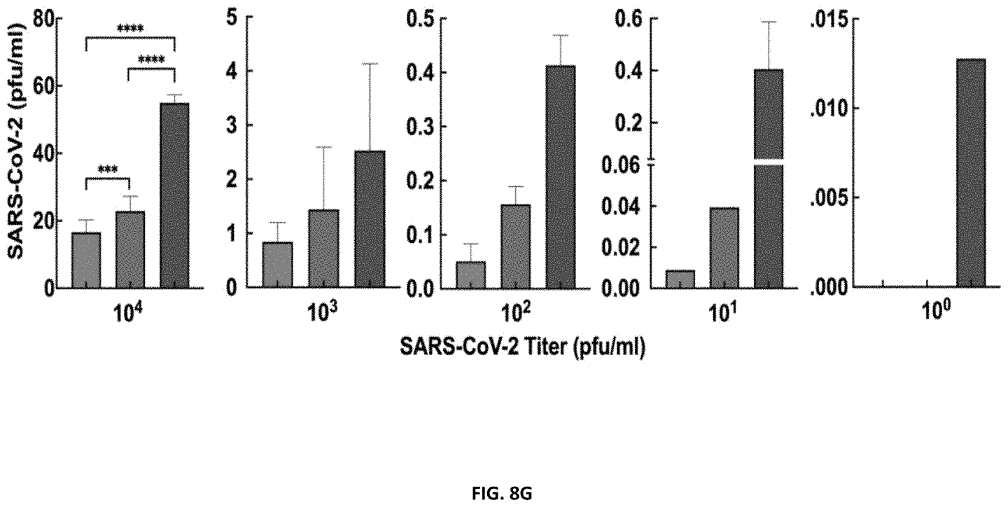

Identification of pathogens by swabbing is, without question, the most critical role swabs play in medicine. Whether identifying bacteria or viruses, ensuring ultrahigh sensitivity is critical for reducing false negative test rates, particularly in patients with low titers or bacterial counts (Pan, et al., Clin. Chem. (2020) 66:794-801; Xiao, et al., J. Med. Virol. (2020) 92(10):1755-1756; Palavecino, E. L., Rapid Methods for Detection of MRSA in Clinical Specimens. In Methicillin-Resistant Staphylococcus Aureus (MRSA) Protocols; Ji, Y., Ed.; Methods Mol. Biol. Humana Press: Totowa, N.J., 2014; pp 71-83; Roth, et al., PLoS One (2016) 11: No. e0159667; Davis, et al. medRxiv (2020) medrxiv.org/content/10.1101/2020.06.09.20124008v4). Decreased false negative rates via increased swab sensitivity can help identify pathogens and increase the accuracy of viral contact tracing and viral RNA identification. Methicillin-resistance Staphylococcus aureus is one of the most fiscally burdensome bacterial infections in clinics and contributes significantly to increased hospitalization time and decreased survival rates (Roth, et al., PLoS One (2016) 11: No. e0159667; Issler-Fisher, et al., Burns (2015) 41:1212-1220). Nasal swabbing with cotton or flocked swabs is a point-of-care method to rapidly identify a MRSA infection (Palavecino, E. L., Rapid Methods for Detection of MRSA in Clinical Specimens. In Methicillin-Resistant Staphylococcus Aureus (MRSA) Protocols; Ji, Y., Ed.; Methods Mol. Biol. Humana Press: Totowa, N.J., 2014; pp 71-83; Schulz, et al., Lab Chip (2020) 20:2549-2561). The relative sensitivity of nanofiber swabs was determined using MRSA as a model bacterium. Serial dilutions of stock MRSA were prepared and directly swabbed with cotton, flocked, and nanofiber swabs. Swabs were then used to directly inoculate an LB agar plate using a spreading method and colonies were counted after an overnight incubation (FIG. 8A). At 10⁶ CFU/ml, the colony density was too high to count for flocked and nanofiber swabs, but at both 10⁵ and 10⁴ CFU/ml the colony density is distinguishably higher in cultures from nanofiber swabs (FIG. 8B). At 10⁵ and 10⁴ CFU/ml, flocked and nanofiber swabs exhibited substantially higher sensitivity compared to cotton swabs, and nanofiber swabs achieved roughly double the colony counts as flocked swabs (FIG. 8C, 8D). Bacterial swabs are typically performed via nose, mouth, or throat where bacteria-containing droplets are absorbed onto the swab and identified by RT-PCR, morphological phenotyping, or quadrant streaking and culture (Wigger, et al., J. Microbiol. Methods (2019) 157:47-49; Luabeya, et al., J. Clin. Microbiol. (2019) 57: e01847-18; Fisher, et al., J. Clin. Virol. (2019) 115:43-46). The ultrahigh absorptivity and release of biological materials could make nanofiber swabs excellent candidates for improved bacteria swabbing in clinics, as they not only improve sensitivity at low bacteria densities but are suitable for each standard postharvest analysis technique.

Similarly, identification of viral pathogens commonly occurs through nose, mouth, or throat swabbing, where sensitive swabs are needed to collect as much virus-containing droplets as possible (Fisher, et al., J. Clin. Virol. (2019) 115:43-46; Abu-Diab, et al., J. Clin. Microbiol. (2008) 46:2414-2417; Tunsjø, et al., APMIS (2015) 123:473-477). Increased sensitivity of SARS-CoV-2 tests result in a decrease in false negative test rates, which is vital in allowing time for treatment and early identification (Woloshin, et al., N. Engl. J. Med. (2020) 383: e38). To compare sensitivity of nanofiber swabs to cotton and flocked swabs, a serial dilution of SARS-CoV-2 was made and each dilution was swabbed by each swab type (FIG. 8E). Swab tips were vortexed in a buffer, and PCR amplification of SARSCoV-2 was carried out. At 10²-10⁴ pfu/ml, each swab type identified SARS-CoV-2 with nanofiber swabs requiring fewer PCR cycles at each titer. At 10¹ pfu/ml, both cotton and flocked swabs had a false negative rate of 66.7%, whereas nanofiber swabs had a 0% false negative rate. At 10⁰ pfu/ml, both cotton and flocked swabs had a 100% false negative rate, while nanofiber swabs had a 66.7% false negative rate (FIG. 8F). Nanofiber swabs exhibited reduced cycle thresholds at every titer concentration and identified SARS-CoV-2 at 10⁰ pfu/ml, a concentration ten times lower than the lowest identifiable titer using cotton or flocked swabs. Similarly, at every titer, higher concentrations of SARS-CoV-2 were detected, after interpolating pfu/ml from cycle threshold values (FIG. 8G). Notably, nanofiber swabs detected roughly 10-fold pfu/ml at 10¹ pfu/ml titer, a range that becomes increasingly hard to detect. Summary data relating cycle thresholds, detected pfu/ml, and false negative/true positive rates are detailed in Tables 2-4. This experiment demonstrated that nanofiber swabs had improved sensitivity and decreased false negative rates compared to cotton and flocked swabs. During the ongoing COVID19 pandemic, implementation of supersensitive nanofiber swabs can identify fragment or low titers characteristic of early stages of SARS-CoV-2 infection. Increasing the sensitivity of testing allows for virus detection in earlier stages of infection, thus reducing spreading and lending precious time to clinicians and patients who may develop severe symptoms.

TABLE 2
SARS-COV-2 detection with cotton swab.

| Titer (pfu/ml) | 10⁴ | 10³ | 10² | 10¹ | 10⁰ |
|---|---|---|---|---|---|
| Cycle | 23.68 ± 0.32 | 28.09 ± 0.70 | 32.25 ± 0.87 | 38.24 ± 0.00 | und |
| Threshold (CT) | | | | | |
| Concentration | 16.60 ± 3.7 | 0.842 ± 0.35 | 0.051 ± 0.03 | 0.001 ± 0.00 | 0.000 ± 0.00 |
| (pfu/ml) | | | | | |
| True Positive | 100% | 100% | 100% | 33.33% | 0.00% |
| Rate (%) | | | | | |
| False Negative | 0% | 0% | 0% | 66.67% | 100% |
| Rate (%) | | | | | |

Mean ± SD.
und = undetected.
Equivalent pfu/ml = 2E+08e^{-0.689}*^{CT}.

TABLE 3
SARS-COV-2 detection with flocked swab.

| Titer (pfu/ml) | 10⁴ | 10³ | 10² | 10¹ | 10⁰ |
|---|---|---|---|---|---|
| Cycle | 23.21 ± 0.26 | 28.00 ± 2.3 | 29.12 ± 2.3 | 34.91 ± 0.00 | und |
| Threshold (CT) | | | | | |
| Concentration | 22.90 ± 4.3 | 1.44 ± 1.2 | 0.920 ± 1.3 | 0.007 ± 0.00 | 0.000 ± 0.00 |
| (pfu/ml) | | | | | |
| True Positive | 100% | 100% | 100% | 33.33% | 0.00% |
| Rate (%) | | | | | |
| False Negative | 0% | 0% | 0% | 66.67% | 100% |
| Rate (%) | | | | | |

Mean ± SD.
und = undetected.
Equivalent pfu/ml = 2E+08e^{-0.689}*^{CT}.

TABLE 4
SARS-COV-2 detection with nanofiber swab.

| Titer (pfu/ml) | 10⁴ | 10³ | 10² | 10¹ | 10⁰ |
|---|---|---|---|---|---|
| Cycle | 21.93 ± 0.06 | 26.57 ± 0.86 | 29.03 ± 0.20 | 33.24 ± 0.80 | 36.05 ± 2.93 |
| Threshold (CT) | | | | | |
| Concentration | 54.95 ± 2.44 | 2.53 ± 1.6 | 0.413 ± 0.06 | 0.025 ± 0.01 | 0.007 ± 0.01 |
| (pfu/ml) | | | | | |
| True Positive | 100% | 100% | 100% | 100% | 66.67% |
| Rate (%) | | | | | |
| False Negative | 0% | 0% | 0% | 0% | 33.33% |
| Rate (%) | | | | | |

Mean ± SD.
und = undetected.
Equivalent pfu/ml = 2E+08e^{-0.689}*^{CT}.

In summary, a new type of swab tipped with cylindrical nanofiber objects produced by gas-foam expanding electrospun membranes around a fixed axis has been developed. Because of the small diameters and hierarchical porous structures, nanofiber swabs showed ultrahigh absorption, far surpassing the absorptive capabilities of cotton and flocked swabs. Additionally, the improved ability of the nanofiber swabs to detect samples from surfaces as well as release absorbed samples is demonstrated. The shape-tunable and morphologically hierarchical swabs showed improved collection and identification of proteins, cells, DNA, bacteria, and viruses. Nanofiber swabs would increase the diagnostic accuracy and assessment of population infection rates during the COVID-19 pandemic. Production of nanofiber swabs can occur at an industrial level, as industrial electrospinning and lyophilization are common, thus making large-scale production of such swabs feasible. These nanofiber swabs have far-reaching potential in medical and forensic fields and serve as an important step toward increasing diagnostic test sensitivity. Although the demand for highly sensitive COVID-19 tests may decrease over time, the need to accurately identify pathogens or proteins in contaminated food samples or identify persons of interest from blood or DNA at a crime scene will always exist. Improving the outcome of specimen identification will help in treatment and prevention of foodborne illnesses, reduce false incarcerations from insensitive DNA analysis, and help forensic teams identify persons of interest from often miniscule biological specimens.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for producing a nanofiber swab, said method comprising:
a) fixing at least one point of a nanofiber mat, wherein said nanofiber mat comprises electrospun nanofibers,
b) expanding the nanofiber mat after step a) by exposing the nanofiber mat to gas bubbles, thereby producing an expanded nanofiber structure, and
c) attaching the expanded nanofiber structure to an end of a handle, thereby producing said nanofiber swab.

2. The method of claim 1, wherein said expanded nanofiber structure has a rounded geometry.

3. The method of claim 1, wherein said expanded nanofiber structure is cylindrical or capsular.

4. The method of claim 1, wherein said expanded nanofiber structure has a rectangular or cubic geometry.

5. The method of claim 1, wherein said gas bubbles are generated as a product of a chemical reaction.

6. The method of claim 5, wherein said chemical reaction is the hydrolysis of sodium borohydride.

7. The method of claim 1, wherein step b) comprises exposing the nanofiber mat to a subcritical fluid and depressurizing.

8. The method of claim 7, wherein said subcritical fluid is subcritical CO₂.

9. The method of claim 1, wherein said nanofiber mat comprises a plurality of aligned nanofibers, random nanofibers, and/or entangled nanofibers.

10. The method of claim 1, further comprising synthesizing said nanofiber mat by electrospinning prior to step a).

11. The method of claim 1, further comprising cutting said nanofiber mat prior to step a) or b).

12. The method of claim 1, wherein said nanofiber mat comprises polycaprolactone (PCL).

13. The method of claim 1, wherein said nanofiber mat comprises a poloxamer.

14. The method of claim 13, wherein said poloxamer is poloxamer 407.

15. The method of claim 1, wherein step a) comprises thermally fixing at least one point of said nanofiber mat.

16. The method of claim 1, wherein step a) comprises fixing at least an entire side of said nanofiber mat.

17. The method of claim 1, further comprising coating the expanded nanofiber structure with a hydrogel.

18. The method of claim 17, wherein said hydrogel comprises gelatin, gelatin methacryloyl (GelMA), and/or chitosan.

19. The method of claim 17, further comprising crosslinking the expanded nanofiber structure and/or hydrogel.

20. The method of claim 19, wherein said crosslinking comprises contacting said expanded nanofiber structure and/or hydrogel to glutaraldehyde.

21. The method of claim 1, wherein step c) comprises applying an adhesive to the handle and inserting the handle into the expanded nanofiber structure.

22. A nanofiber swab produced by the method of claim 1.

23. A nanofiber swab comprising an expanded nanofiber structure and a handle, wherein said expanded nanofiber structure is attached to the terminus of said handle.

24. A method of collecting a sample from a surface or a subject, said method comprising contacting a nanofiber swab of claim 23 with a site in or on the surface or subject and collecting the sample from the site via the expanded nanofiber structure.

25. The method of claim 24, wherein the sample comprises a bacterium, a virus, or a cell.

26. The method of claim 24, wherein the sample comprises a nucleic acid or a polypeptide.
